(19) United States
(12) Patent Application Publication
(10) Pub. No.: US 2023/0207114 A1
(43) Pub. Date: Jun. 29, 2023

(54) BIOLOGICAL INFORMATION ANALYSIS SYSTEM, NON-TRANSITORY COMPUTER READABLE MEDIUM AND BIOLOGICAL INFORMATION ANALYSIS METHOD

(71) Applicant: NIPPON TELEGRAPH AND TELEPHONE CORPORATION, Chiyoda-ku (JP)

(72) Inventors: Takayuki OGASAWARA, Musashino-shi (JP); Kentaro TANAKA, Musashino-shi (JP); Shingo TSUKADA, Musashino-shi (JP); Masumi YAMAGUCHI, Musashino-shi (JP); Toichiro GOTO, Musashino-shi (JP)

(73) Assignee: NIPPON TELEGRAPH AND TELEPHONE CORPORATION, Tokyo (JP)

(21) Appl. No.: 17/925,767

(22) PCT Filed: May 21, 2020
(86) PCT No.: PCT/JP2020/020157

§ 371 Date: Nov. 16, 2022

(51) Int. Cl. G16H 40/67; G16H 15/00

(52) U.S. Cl. CPC G16H 40/67; G16H 15/00

(57) ABSTRACT

A biological information analysis system includes a data reception unit that receives time-series data of biological information from a sensor terminal that acquires biological information, a storage unit that stores the received time-series data, a first representative value acquisition unit that acquires a first representative value based on data in a predetermined period among the received time-series data, and a second representative value acquisition unit that acquires a second representative value based on a plurality of first representative values consecutive on a time axis. In a case where an error occurs in communication between the data reception unit and the sensor terminal and a predetermined condition is satisfied, the first representative value acquisition unit uses pre-error time-series data transmitted before the error occurs and stored in the storage unit and post-error time-series data transmitted from the sensor terminal after the error occurs, to acquire the first representative value.

### TECHNICAL FIELD

The present invention relates to a biological information analysis system, a non-transitory computer readable medium, and a biological information analysis method.

### BACKGROUND ART

In the field of medical treatment and long-term care, a system has been proposed for monitoring biological information of a patient in 24-hour daily activity using a sensor and performing appropriate treatment (see, for example, NPL 1). Such a system includes, for example, a sensor terminal that acquires data of biological information, a relay terminal that acquires the data of the biological information from the sensor terminal and relays the data to another apparatus, and an external terminal that performs, for example, analysis based on the data of the biological information. In such a system, in order to reduce a load of traffic in a network between the relay terminal and the external terminal, the relay terminal performs downsampling on the data of the biological information. Examples of the downsampling include a method of periodically thinning out data, and a method of periodically calculating a statistical value (for example, an average value) of data during a certain period.

Further, there are a plurality of types of technologies for transmitting data of biological information that is a downsampling target from a sensor terminal to a relay terminal. For example, there is a technology of transmitting the data in real time from the sensor terminal to the relay terminal using a wireless module. For example, there is also a technology for storing data of biological information once in a built-in memory included in a sensor terminal and then transmitting the data to a relay terminal at a predetermined timing (see, for example, NPL 2).

### CITATION LIST

### Non Patent Literature

[Non-Patent Literature 1] “Efforts for rehabilitation support applying wearable material Hitoe,” NTT Technical Journal
[Non-Patent Literature 2] “Development of a low-power, compact wearable sensor that enables measurement of electrocardiogram, acceleration, temperature, and humidity for smart healthcare”<URL: https:/www.ntt.co.jp/news2019/1911/191108a.html>

### SUMMARY OF THE INVENTION

### Technical Problem

When the data is transmitted from the sensor terminal to the relay apparatus, communication may fail due to an influence of noise or interference in a process of wireless communication (for example, Bluetooth Low Energy: BLE). Thereafter, when the transmission is restarted by reconnecting the communication, processing using the transmitted data may not be performed appropriately. For example, it is difficult to determine whether the data arriving at the relay terminal after the restart of the transmission should be treated as a set of data with data transmitted before the restart or as a set of data different from the data transmitted before the restart, and erroneous treatment may occur. When erroneous treatment occurs, there is concern that accuracy of the data is degraded. Specific examples of data to be treated as a set of data include data of biological information of the same user, and data used to obtain the same statistical value. Specific examples of the data that should be treated as a set of data different from the data transmitted before restart include data of biological information of other users, data of biological information newly transmitted after normal communication has ended once, and data used to obtain different statistical values.

Further, in order to prevent the erroneous treatment, when communication fails, some data transmitted before restart of transmission may be processed in such a manner that the data is not used for calculation of statistical values. However, in such processing, some of the data is missing, and thus there is concern that accuracy of subsequent analysis or the like may be degraded.

In view of the above circumstances, an object of the present invention is to provide a technology capable of curbing accuracy degradation and missing of data even when a failure occurs in data communication.

### Means for Solving the Problem

According to an aspect of the present invention, a biological information analysis system includes: a data reception unit configured to receive time-series data of biological information from a sensor terminal having a biological sensor configured to acquire biological information; a storage unit configured to store the received time-series data; a first representative value acquisition unit configured to acquire a first representative value based on data in a predetermined period among the received time-series data; and a second representative value acquisition unit configured to acquire a second representative value based on a plurality of first representative values consecutive on a time axis. The first representative value is one of the plurality of first representative values. In a case where an error occurs in communication between the data reception unit and the sensor terminal and a predetermined condition is satisfied, the first representative value acquisition unit uses pre-error time-series data transmitted before the error occurs and stored in the storage unit and post-error time-series data transmitted from the sensor terminal after the error occurs, to acquire the first representative value.

According to an aspect of the present invention, a non-transitory computer readable medium has a program stored therein for causing a computer to function as the biological information analysis system.

According to an aspect of the present invention, a biological information analysis method includes: receiving time-series data of biological information from a sensor terminal having a biological sensor configured to acquire biological information; storing the received time-series data; acquiring a first representative value based on data in a predetermined period among the received time-series data; and acquiring a second representative value based on a plurality of first representative values consecutive on a time axis. The first representative value is one of the plurality of first representative values. In the acquiring of the first representative value, in a case where an error occurs in communication between the data reception unit and the sensor terminal and a predetermined condition is satisfied, the first representative value is acquired using pre-error time-series data transmitted before the error occurs and stored in the storage unit and post-error time-series data transmitted from the sensor terminal after the error occurs.

### Effects of the Invention

According to the present invention, it is possible to curb accuracy degradation and missing of data even when a failure occurs in data communication.

### BRIEF DESCRIPTION OF DRAWINGS

FIG. 1 is a block diagram illustrating functions of a biological information analysis apparatus according to a first embodiment of the present invention.

FIG. 2 is a block diagram illustrating a hardware configuration of the biological information analysis apparatus according to the first embodiment.

FIG. 3 is a flowchart illustrating a biological information analysis method according to the first embodiment.

FIG. 4 is a diagram illustrating processing for calculating a representative value according to the first embodiment.

FIG. 5 is a diagram illustrating a configuration of a biological information analysis system according to the first embodiment.

FIG. 6 is a block diagram illustrating a configuration of the biological information analysis system according to the first embodiment.

FIG. 7 is a sequence diagram illustrating operations of the biological information analysis system according to the first embodiment.

FIG. 8 is a block diagram illustrating functions of a biological information analysis apparatus according to a second embodiment.

FIG. 9 is a diagram illustrating adjustment processing according to the second embodiment.

FIG. 10 is a diagram illustrating a final representative value at a data end according to the second embodiment.

FIG. 11 is a sequence diagram illustrating operations of the biological information analysis system according to the second embodiment.

FIG. 12 is a diagram illustrating effects of the biological information analysis apparatus according to the second embodiment.

FIG. 13 is a block diagram illustrating functions of a biological information analysis apparatus according to a third embodiment of the present invention.

FIG. 14 is a diagram illustrating processing for calculating a representative value according to the third embodiment.

FIG. 15 is a block diagram illustrating a configuration of a biological information analysis system according to the third embodiment.

FIG. 16 is a sequence diagram illustrating operations of the biological information analysis system according to the third embodiment.

FIG. 17 is a block diagram illustrating functions of a biological information analysis apparatus according to a fourth embodiment of the present invention.

FIG. 18 is a diagram illustrating processing for calculating a representative value according to the fourth embodiment.

FIG. 19 is a block diagram illustrating a configuration of a biological information analysis system according to the fourth embodiment.

FIG. 20 is a sequence diagram illustrating operations of the biological information analysis system according to the fourth embodiment.

FIG. 21 is a block diagram illustrating functions of a biological information analysis apparatus according to a fifth embodiment of the present invention.

FIG. 22 is a diagram illustrating processing for calculating a representative value according to the fifth embodiment.

FIG. 23 is a block diagram illustrating a configuration of a biological information analysis system according to the fifth embodiment.

FIG. 24 is a sequence diagram illustrating operations of the biological information analysis system according to the fifth embodiment.

FIG. 25 is a block diagram illustrating functions of a biological information analysis apparatus according to a modification example of the fifth and sixth embodiments of the present invention.

FIG. 26 is a sequence diagram illustrating operations of a biological information analysis system according to the sixth embodiment.

### DESCRIPTION OF EMBODIMENTS

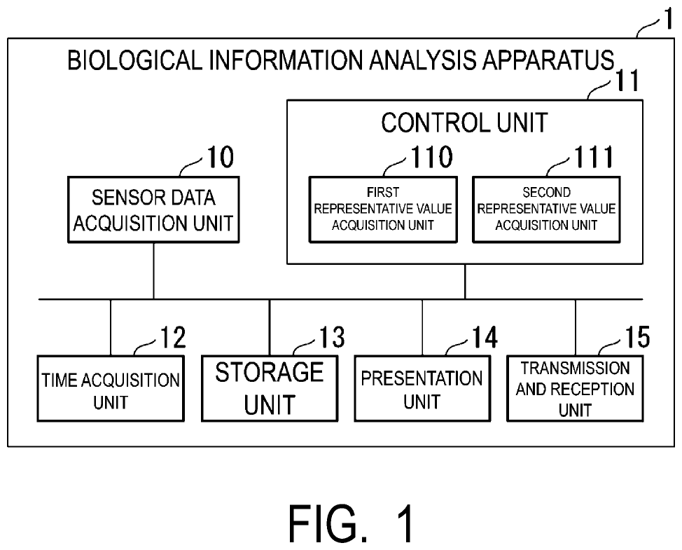

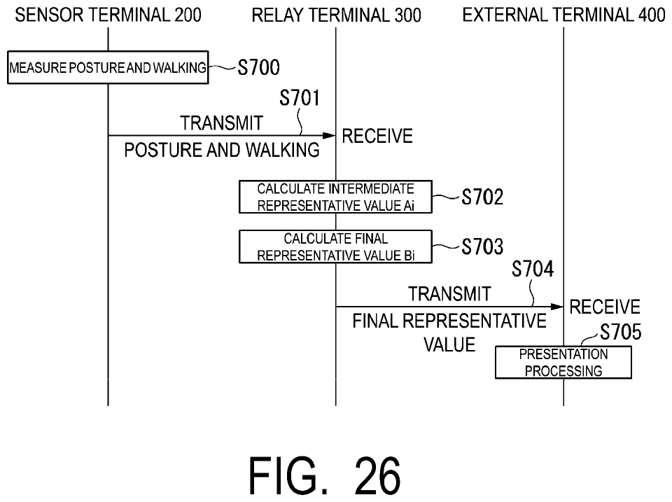

Hereinafter, preferred embodiments of the present invention will be described in detail with reference to FIGS. 1 to 26.

### First Embodiment

First, an overview will be given for a configuration of a biological information analysis apparatus 1 according to a first embodiment of the present invention.

FIG. 1 is a block diagram illustrating a functional configuration of the biological information analysis apparatus 1.

Functional Blocks of Biological Information Analysis Apparatus A biological information analysis apparatus 1 includes a sensor data acquisition unit (sensor data acquisitor) 10, a control unit (controller) 11, a time acquisition unit (time acquisitor) 12, a storage unit (storage) 13, a presentation unit (presenter) 14, and a transmission and reception unit (transceiver) 15.

The sensor data acquisition unit 10 acquires biological information of a user measured by a sensor 105, which will be described below, mounted on the user. More specifically, the sensor data acquisition unit 10 calculates a heart rate from an electrocardiographic waveform based on an electrocardiographic potential measured by a heart rate monitor, when, for example, the heart rate monitor is mounted on the user as the sensor 105. Further, when an acceleration sensor is mounted on the user as the sensor (biological sensor) 105, the sensor data acquisition unit 10 converts an analog acceleration signal measured by the acceleration sensor to a digital signal at a predetermined sampling rate.

The sensor data acquisition unit 10 outputs time-series data in which the heart rate or an acceleration signal of digital data and a measurement time at which the acceleration signal has been acquired by the sensor 105 are associated with each other. In this example, the heart rate and acceleration data are biological information. The time-series data of the biological information measured by the sensor data acquisition unit 10 is stored in the storage unit 13 to be described below.

The control unit 11 includes a first representative value acquisition unit (first representative value acquisitor) 110 and a second representative value acquisition unit (second representative value acquisitor) 111. The control unit 11 acquires a statistical representative value of the time-series data of the biological information of the user stored in the storage unit 13. In the present embodiment, the statistical representative value of the time-series data of the biological information of the user is acquired through calculation. In the present embodiment, in order to acquire the statistical representative value of the time-series data of the biological information of the user, for example, processing using a look-up table may be used in addition to the calculation. In the present embodiment, the control unit 11 calculates a representative value of the biological information in two steps. The control unit 11 may calculate, for example, an average value or a proportion of the biological information at regular intervals as the representative value of the biological information.

The first representative value acquisition unit 110 calculates a first representative value (hereinafter referred to as an “intermediate representative value”) that is an intermediate representative value of the biological information in each preset period from the time-series data of the biological information of the user. The calculated intermediate representative value is stored in the storage unit 13. More specifically, the first representative value acquisition unit 110 calculates, for example, an intermediate representative value indicating an average value every 60 seconds in the time-series data of the biological information acquired by the sensor data acquisition unit 10.

The second representative value acquisition unit 111 calculates a second representative value (hereinafter, a “final representative value”), which is a final representative value of the time-series data of the biological information, based on one intermediate representative value or a plurality of consecutive intermediate representative values. The calculated final representative value is stored in the storage unit 13.

More specifically, the second representative value acquisition unit 111 calculates one final representative value using, for example, five consecutive intermediate representative values. For example, the second representative value acquisition unit 111 calculates a representative value in a period of five minutes as the final representative value, based on the five consecutive intermediate representative values calculated at intervals of 60 seconds. The number of consecutive intermediate representative values to be used when the second representative value acquisition unit 111 calculates the final representative value can be set freely.

The time acquisition unit 12 acquires a reference time to be used in the biological information analysis apparatus 1. The time acquisition unit 12 may acquire, for example, time information from a clock 107 provided in the biological information analysis apparatus 1 or may acquire time information from a time server (not illustrated). The time information acquired by the time acquisition unit 12 is used when the sensor data acquisition unit 10 performs sampling of the biological information or is used when the first representative value acquisition unit 110 calculates a period in the calculation of the intermediate representative value.

The storage unit 13 stores the time-series data of the biological information of the user measured by the sensor data acquisition unit 10. Further, the storage unit 13 stores the intermediate representative value calculated in each preset period by the first representative value acquisition unit 110. Further, the storage unit 13 stores the final representative value calculated by the second representative value acquisition unit 111.

The presentation unit 14 presents a representative value calculated by the control unit 11. More specifically, the presentation unit 14 displays the final representative value of the biological information on a display apparatus 109 to be described below. Further, the presentation unit 14 generates and presents information for supporting the user based on the final representative value. The presentation unit 14 may output the information for supporting the user to an operating apparatus (not illustrated) that is implemented by the display apparatus 109, an audio output apparatus, a light source, an actuator, a heating device, or the like.

The transmission and reception unit 15 receives sensor data indicating biological information measured by the sensor 105 to be described below. Further, the transmission and reception unit 15 can transmit the final representative value of the biological information by the control unit 11 to the outside via a communication network.

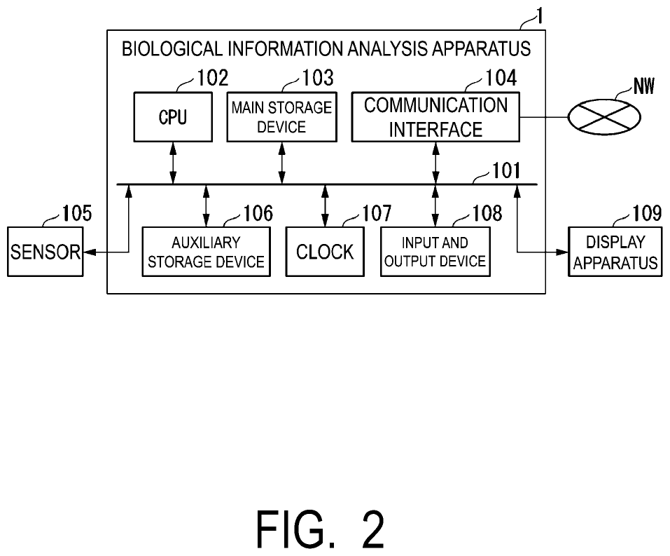

Hardware Configuration of Biological Information Analysis Apparatus Next, an example of a hardware configuration of the biological information analysis apparatus 1 having the above-described functions will be described with reference to the block diagram of FIG. 2.

As illustrated in FIG. 2, the biological information analysis apparatus 1 can be implemented, by, for example, a computer including a CPU 102, a main storage device 103, a communication interface 104, an auxiliary storage device 106, the clock 107, and an input and output device 108 connected via a bus 101, and a program for controlling such hardware resources. The sensor 105 and the display apparatus 109 that are provided externally are connected to the biological information analysis apparatus 1 via the bus 101.

The main storage device 103 stores in advance a program for the CPU 102 to perform various controls or calculations. The CPU 102 and the main storage device 103 achieve functions of the biological information analysis apparatus 1 including the control unit 11 illustrated in FIG. 1.

The communication interface 104 is an interface circuit for performing communication with various external electronic devices via the communication network NW.

As the communication interface 104, for example, a calculation interface and an antenna corresponding to a wireless data communication standard such as Long Term Evolution (LTE), 3G, wireless local area network (LAN), or Bluetooth (trade name) may be used. The communication interface 104 implements the transmission and reception unit 15 described with reference to FIG. 1.

The sensor 105 is implemented by, for example, a heart rate monitor, an electrocardiograph, or a sensor such as an acceleration sensor. The sensor 105 is mounted on the user over a preset measurement period and measures biological information such as a heart rate or acceleration of the user.

The auxiliary storage device 106 includes a readable and writable storage medium, and a drive apparatus for reading and writing various types of information such as programs or data from and to the storage medium. In the auxiliary storage device 106, a hard disk or a semiconductor memory such as a flash memory can be used as the storage medium.

The auxiliary storage device 106 includes a storage area for storing the time-series data of the biological information measured by the sensor 105, and a program storage area for storing a program for the biological information analysis apparatus 1 to perform processing for analyzing the biological information. The auxiliary storage device 106 implements the storage unit 13 described with reference to FIG. 1. Further, for example, a backup area for backing up the above-described data, programs, and the like may be included.

The clock 107 includes, for example, a built-in clock built in a computer, and measures time. Time information obtained by the clock 107 is used for sampling of biological information or processing for calculating the representative value. The time information obtained by the clock 107 is acquired by the time acquisition unit 12 described with reference to FIG. 1.

The input and output device 108 includes an I/O terminal for inputting a signal from an external device such as the sensor 105 or the display apparatus 109 or outputting a signal to the external device.

The display apparatus 109 functions as the presentation unit 14 of the biological information analysis apparatus 1. The display apparatus 109 is implemented by a liquid crystal display or the like. Further, the display apparatus 109 constitutes the operating apparatus that outputs user support information generated based on the final representative value of the biological information.

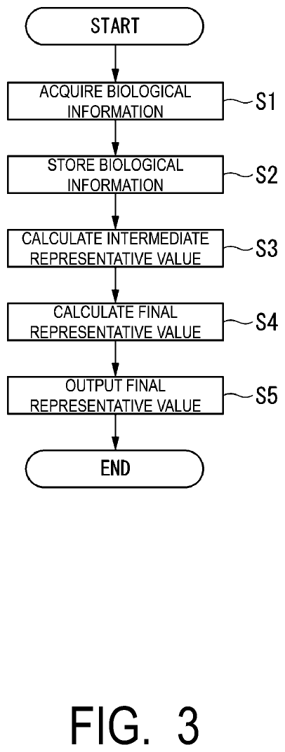

Biological Information Analysis Method Next, operations of the biological information analysis apparatus 1 having the above-described configuration will be described with reference to a flowchart of FIG. 3. First, the following processing is executed in a state in which the sensor 105 is mounted on the user.

The sensor data acquisition unit 10 acquires the biological information measured by the sensor 105 mounted on the user (step S1). More specifically, the sensor data acquisition unit 10 acquires biological information and outputs time-series data in which the biological information and the measurement time are associated with each other. Then, the time-series data of the biological information is stored in the storage unit 13 (step S2).

Then, the first representative value acquisition unit 110 calculates an intermediate representative value of the time-series data of the biological information measured in step S1 (step S3). More specifically, the first representative value acquisition unit 110 calculates an average value of the time-series data of the biological information every preset period, such as every 60 seconds.

Then, after a predetermined time has elapsed, the second representative value acquisition unit 111 calculates the final representative value of the time-series data of the biological information based on the intermediate representative value calculated in step S3 (step S4). More specifically, the second representative value acquisition unit 111 calculates a representative value of a plurality of consecutive intermediate representative values set in advance as the final representative value. For example, the second representative value acquisition unit 111 may calculate the average value of these intermediate representative values after the five consecutive intermediate representative values are calculated in step S3. Thereafter, the control unit 11 outputs the final representative value calculated in step S4 (step S5). The control unit 11 may output the intermediate representative value calculated in step S3 together with the final representative value.

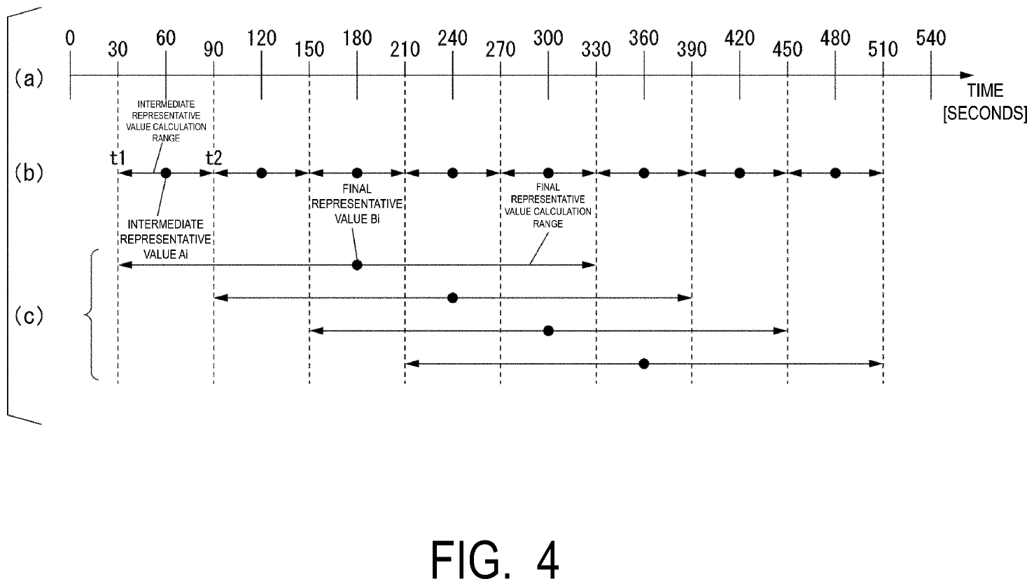

FIG. 4 is a diagram for explaining an example of the processing for calculating a representative value in the control unit 11. An upper part (a) illustrated in FIG. 4 illustrates a data string of the biological information acquired by the sensor data acquisition unit 10 together with an elapsed time. A middle part (b) illustrates a string of intermediate representative values, and a measurement period of biological data (which may be hereinafter referred to as a “calculation period”) that is a basis for calculation of each intermediate representative value. A lower part (c) illustrates a string of final representative values and a period of a calculation range.

Here, an intermediate representative value Aᵢ indicates an i-th calculated intermediate representative value, and is expressed in the form of a matrix. When a sum of the measured values of the biological information in any period is Sᵢ and the number of measured values is Nᵢ, the intermediate representative value Aᵢ is expressed by Equation (1) below.

$$\begin{matrix}[ Math . 1 ] & \\ {A}_{i} = ( {S}_{i} / {N}_{i} ) = [ {\underbrace{{\sum }_{t 1 = 60 i}^{t 2 = 60 i + 30} {a}_{t}}}_{{S}_{i}} / {\underbrace{\{ ( t2 - t1 ) / f \}}}_{{N}_{i}} ] & ( 1 )\end{matrix}$$

In Equation (1) above, at is a measured value of biological information at a measurement time t, and t1 and t2 are a starting point time and an ending point time of a period set for calculation of an intermediate representative value, respectively. Further, in Equation (1), a length of the calculation period is 60 seconds, and the biological information aₜ during that period is sampled in a 1/f cycle. In the present embodiment, sliding occurs by one calculation period, that is, by 60 seconds, each time i increases by one.

The final representative value Bᵢ is expressed by Equation (2) below.

$$\begin{matrix}[ Math . 2 ] & \\ {B}_{i} = \frac{{S}_{i - 2} + {S}_{i - 1} + {S}_{i} + {S}_{i + 1} + {S}_{i + 2}}{{N}_{i - 2} + {N}_{i - 1} + {N}_{i} + {N}_{i + 1} + {N}_{i + 2}} & ( 2 )\end{matrix}$$

Functions of the biological information analysis apparatus 1 described above may be configured not only in one computer but also in a plurality of computers communicatively connected via a communication network in a distributive manner.

Biological Information Analysis System

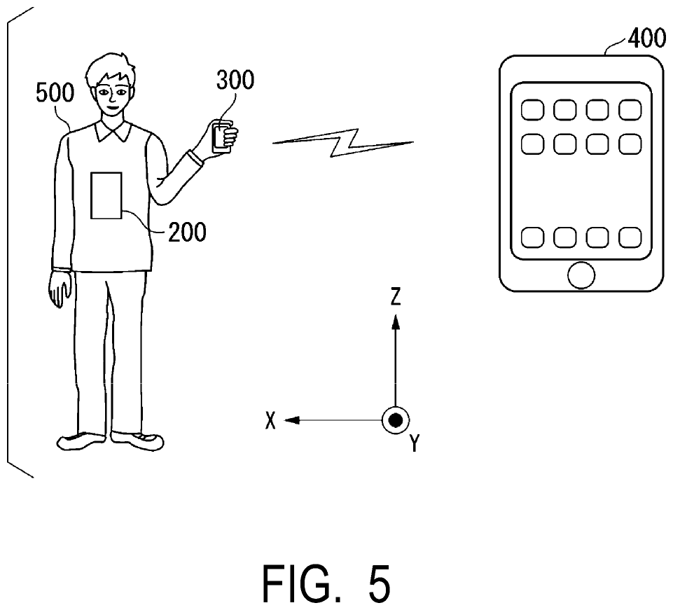

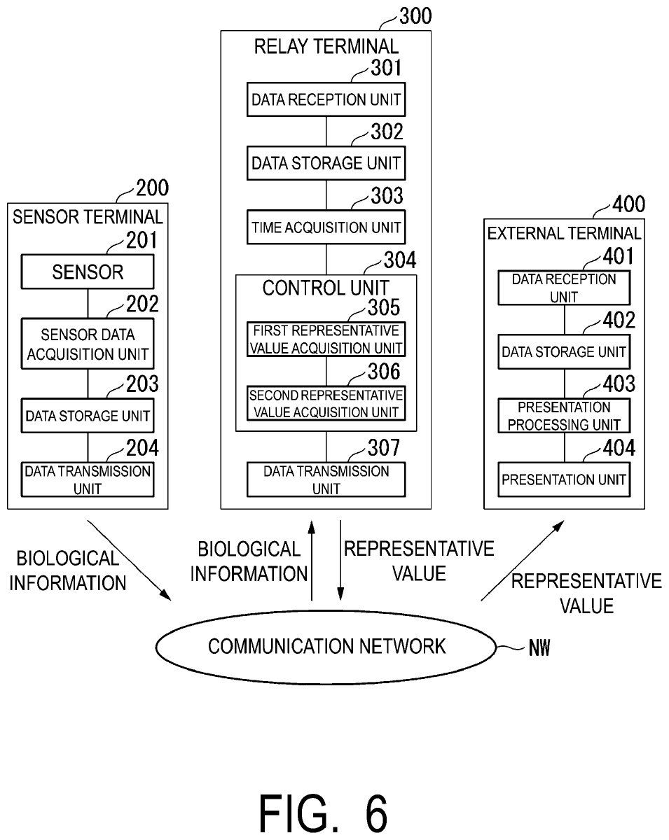

Next, a biological information analysis system specifically constituting the biological information analysis apparatus 1 according to the present invention will be described with reference to FIGS. 5 and 6.

The biological information analysis system includes, for example, a sensor terminal 200 mounted on the user 500, a relay terminal 300, and an external terminal 400, as illustrated in FIG. 5. All or any of the sensor terminal 200, the relay terminal 300, and the external terminal 400 includes functions of the biological information analysis apparatus 1 such as the control unit 11 described with reference to FIG. 1. Hereinafter, a description will be given for a case in which the relay terminal 300 includes the control unit 11 described with reference to FIG. 1.

Functional Blocks of Sensor Terminal

The sensor terminal 200 includes a sensor (biological sensor) 201, a sensor data acquisition unit 202, a data storage unit 203, and a data transmission unit 204. The sensor terminal 200 is disposed on the trunk of the body of the user 500, for example, and measures biological information over a plurality of time cycles. The sensor terminal 200 transmits the measured biological information of the user 500 to the relay terminal 300 via the communication network NW.

The sensor 201 is implemented by a heart rate monitor, an acceleration sensor, or the like. As illustrated in FIG. 5, for example, three axes of the acceleration sensor included in the sensor 201 are provided as an X axis in parallel with a left-right direction of the body, a Y axis in parallel with a front-back direction of the body, and a Z axis in parallel with an up-down direction of the body. The sensor 201 corresponds to the sensor 105 described with reference to FIG. 2.

The sensor data acquisition unit 202 acquires the biological information measured by the sensor 201. More specifically, the sensor data acquisition unit 202 performs removal of noise from the acquired biological information as necessary and performs sampling processing to obtain time-series data in biological information of a digital signal. The sensor data acquisition unit 202 corresponds to the sensor data acquisition unit 10 described with reference to FIG. 1.

The data storage unit 203 stores the biological information detected by the sensor 201 or time-series data of the biological information according to a digital signal obtained through processing in the sensor data acquisition unit 202. The data storage unit 203 corresponds to the storage unit 13 (FIG. 1).

The data transmission unit 204 transmits the time-series data of the biological information stored in the data storage unit 203 to the relay terminal 300 via the communication network NW. The data transmission unit 204 includes, for example, a communication circuit for performing wireless communication corresponding to a wireless data communication standard such as LTE, 3G, wireless LAN, or Bluetooth (trade name). The data transmission unit 204 corresponds to the transmission and reception unit 15 (FIG. 1).

Functional Blocks of Relay Terminal

The relay terminal 300 includes a data reception unit 301, a data storage unit 302, a time acquisition unit 303, a control unit 304, and a data transmission unit 307. The relay terminal 300 obtains the statistical representative value step by step from the time-series data of the biological information of the user 500 received from the sensor terminal 200. Further, the relay terminal 300 transmits the calculated representative value to the external terminal 400.

The relay terminal 300 is implemented by a smartphone, a tablet, a notebook computer, or the like.

The data reception unit 301 receives the time-series data of the biological information from the sensor terminal 200 via the communication network NW. The data reception unit 301 corresponds to the transmission and reception unit 15 (FIG. 1).

The data storage unit 302 stores the biological information of the user 500 received by the data reception unit 301 or the representative value of the biological information acquired by the control unit 304. The data storage unit 302 corresponds to the storage unit 13 (FIG. 1).

The time acquisition unit 303 acquires time information to be used in processing of analyzing biological information in the control unit 304, from a built-in clock (clock 107). The time acquisition unit 303 corresponds to the time acquisition unit 12 described with reference to FIG. 1.

The control unit 304 includes a first representative value acquisition unit 305 and a second representative value acquisition unit 306.

The control unit 304 obtains, step by step, the statistical representative value such as an average value of time-series data of the biological information of the user 500 received by the data reception unit 301. The control unit 304 corresponds to the control unit 11 described with reference to FIG. 1.

The first representative value acquisition unit 305 calculates an intermediate representative value from the time-series data of the biological information of the user 500 every preset period, such as every 60 seconds. The calculated intermediate representative value is stored in the data storage unit 302.

The second representative value acquisition unit 306 calculates a final representative value from a plurality of consecutive intermediate representative values.

The first representative value acquisition unit 305 and the second representative value acquisition unit 306 correspond to the first representative value acquisition unit 110 and the second representative value acquisition unit 111 described with reference to FIG. 1, respectively.

The data transmission unit 307 transmits the final representative value calculated by the second representative value acquisition unit 306 to the external terminal 400 via the communication network NW. The data transmission unit 307 corresponds to the transmission and reception unit 15 (FIG. 1). The data transmission unit 307 may transmit an intermediate representative value together with the final representative value.

Functional Blocks of External Terminal

The external terminal 400 includes a data reception unit (data receiver) 401, a data storage unit 402, a presentation processing unit 403, and a presentation unit 404. The external terminal 400 presents the final representative value of the biological information of the user 500 received from the relay terminal 300 via the communication network NW. The external terminal 400 presents support information for the user 500 generated based on the calculated final representative value.

The external terminal 400 is implemented by a smartphone, a tablet, a notebook computer, or the like, as with the relay terminal 300. The external terminal 400 includes a display apparatus that displays the received final representative value, and an operating apparatus (not illustrated) that outputs information for supporting the user 500 generated based on the calculated final representative value. Examples of the operating apparatus included in the external terminal 400 include a display apparatus, an audio output apparatus, a light source, an actuator, and a heating device.

As the audio output apparatus, for example, a speaker or a musical instrument may be used. As the light source, a light emitting diode (LED) or a light bulb may be used. As the actuator, a vibrator, a robot arm, or an electrotherapy device may be used. Further, as the heating device, a heater, a Pelche element, or the like may be used.

The data reception unit 401 receives the final representative value of the biological information from the relay terminal 300 via the communication network NW. The data reception unit 401 corresponds to the transmission and reception unit 15 (FIG. 1).

The data storage unit 402 stores the final representative value of the biological information received by the data reception unit 401. The data storage unit 402 corresponds to the storage unit 13 (FIG. 1).

The presentation processing unit 403 generates the support information for the user 500 based on the received final representative value. The presentation processing unit 403 corresponds to the presentation unit 14 described with reference to FIG. 1.

The presentation unit 404 presents the support information for the user 500, based on the presentation of the final representative value and an instruction from the presentation processing unit 403. More specifically, the final representative value or the support information may be displayed through character information or a graph on the display apparatus included in the external terminal 400, or the support information may be output through an alert sound or the like from a speaker (not illustrated) included in the external terminal 400. Further, the presentation unit 404 can present the support information using a method that can be recognized by the user 500, such as vibration or light. The presentation unit 404 corresponds to the presentation unit 14 described with reference to FIG. 1.

Thus, the biological information analysis system according to the present invention has a configuration in which the respective functions of the biological information analysis apparatus 1 are distributed to the sensor terminal 200, the relay terminal 300, and the external terminal 400, and performs processing regarding calculation of the representative value from the measurement of the biological information of the user 500 and the presentation of the final representative value in a distributive manner.

Operation Sequence of Biological Information Analysis System

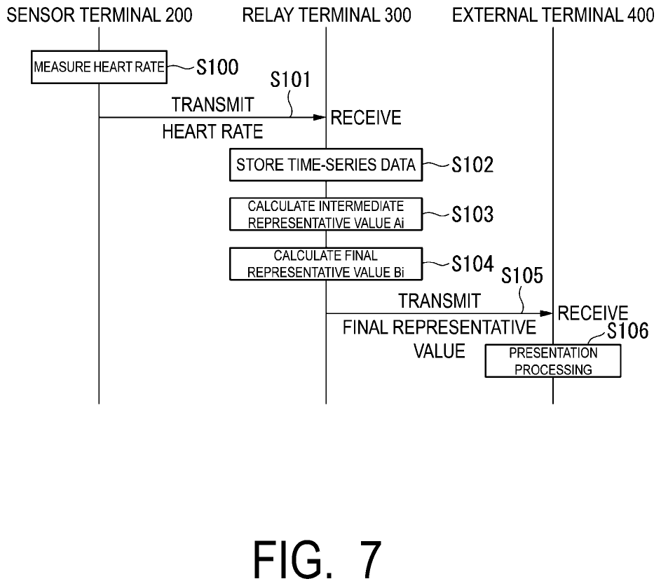

Next, operations of the biological information analysis system having the above-described configuration will be described with reference to a sequence diagram of FIG. 7. In the following description, a representative value of the heart rate of the user 500, which is an example of biological information, is obtained.

As illustrated in FIG. 7, first, the sensor terminal 200 mounted on the user 500 measures the heart rate of the user 500 (step S100). More specifically, the sensor 201 configured of a heart rate monitor measures an electrocardiographic potential of the user 500. The sensor data acquisition unit 202 acquires the heart rate of the user 500 from an electrocardiographic waveform based on the electrocardiographic potential.

Then, the sensor terminal 200 transmits time-series data of the heart rate of the user 500 to the relay terminal 300 via the communication network NW (step S101). When the relay terminal 300 receives the time-series data of the heart rate from the sensor terminal 200, the relay terminal 300 stores the received time-series data in the data storage unit 302 (step S102). The relay terminal 300 calculates an intermediate representative value from the received time-series data in each preset period (step S103). More specifically, the first representative value acquisition unit 305 uses Equation (2) described above to calculate the intermediate representative value Aᵢ every 60 seconds.

Then, the second representative value acquisition unit 306 calculates a final representative value from a plurality of consecutive intermediate representative values after the lapse of a predetermined time (step S104). More specifically, the second representative value acquisition unit 306 uses five consecutive intermediate representative values to calculate the final representative value Bᵢ using Equation (2) above.

Thus, the intermediate representative value is used when the acquired representative value of the time-series data of the biological information is obtained, making it possible to reduce, for example, 300 pieces of data to 10 intermediate representative values as elements of a matrix and then calculate the final representative value. Further, because it is possible to repeat the calculation of the final representative value Bᵢ at a previous stage before data transfer, the same effects as those when a moving average is multiplied and then downsampling is performed on data at regular periods (every 60 seconds in the present embodiment) can be obtained. Thus, the effect of reduction of an amount of data to be transferred can be obtained.

Returning to FIG. 7, the relay terminal 300 then transmits the final representative value of the time-series data of the heart rate of the user 500 to the external terminal 400 via the communication network NW (step S105). When the external terminal 400 receives the final representative value, the external terminal 400 executes the presentation processing (step S106). That is, the external terminal 400 causes the display apparatus to display the final representative value. Further, the external terminal 400 generates the support information for the user 500 based on the final representative value and causes the display apparatus or the like to display the support information.

As described above, the biological information analysis apparatus 1 according to the first embodiment calculates the intermediate representative value from the time-series data of the biological information in each preset period, and calculates the final representative value based on the plurality of consecutive intermediate representative values. Thus, it is possible to simultaneously realize data reduction and summary of the measured biological information in parallel.

### Second Embodiment

Next, a second embodiment of the present invention will be described. In the following description, the same configurations as those of the first embodiment described above are denoted by the same reference signs, and description thereof will be omitted.

In the first embodiment, the description has been given for a case in which the second representative value acquisition unit 111 calculates the final representative value based on the preset number of intermediate representative values. On the other hand, in the second embodiment, a control unit 11A further includes an adjustment unit 112. The adjustment unit 112 changes the number of intermediate representative values that the second representative value acquisition unit 111 uses to calculate the final representative value, based on the number of intermediate representative values that can be secured in the past or future. Hereinafter, a configuration different from that of the first embodiment will be mainly described.

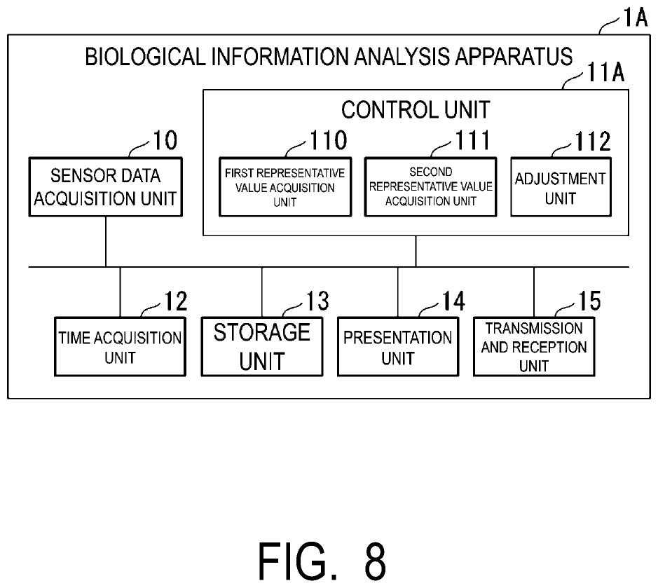

As illustrated in FIG. 8, a biological information analysis apparatus 1A includes a control unit 11A. The control unit 11A includes a first representative value acquisition unit 110, a second representative value acquisition unit 111, and an adjustment unit 112. Other functional configurations included in the biological information analysis apparatus 1A are the same as those in the first embodiment.

The adjustment unit 112 determines the number of intermediate representative values that the second representative value acquisition unit 111 uses for calculation of the final representative value, based on the number of intermediate representative values already calculated. More specifically, each time the second representative value acquisition unit 111 calculates the final representative value, the adjustment unit 112 monitors whether there are a sufficient number of intermediate representative values to calculate the final representative value. For example, the adjustment unit 112 counts the five consecutive intermediate representative values required when the second representative value acquisition unit 111 calculates the final representative value Bᵢ using Equation (2) described above.

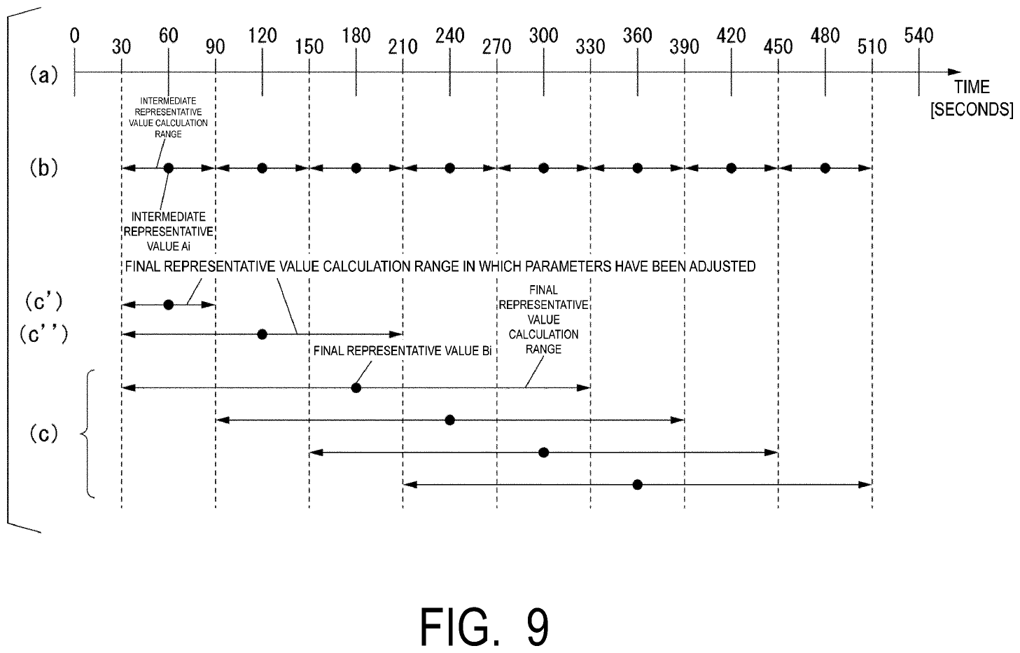

More specifically, as illustrated in FIG. 9, in order for the second representative value acquisition unit 111 to calculate the final representative value Bᵢ illustrated in a lower part (c), five consecutive intermediate representative values Aᵢ are required. When the second representative value acquisition unit 111 calculates, for example, the final representative value Bᵢ according to a setting or an external signal at 120 seconds after start of measurement of biological information, five intermediate representative values Aᵢ are required but only one intermediate representative value is calculated.

In this case, the adjustment unit 112 adjusts the number of intermediate representative values Aᵢ that the second representative value acquisition unit 111 requires to calculate the final representative value. The adjustment unit 112 adopts the smaller of the number of intermediate representative values Aᵢ already calculated at a point in time when the second representative value acquisition unit 111 calculates the final representative value and the number of intermediate representative values Aᵢ to be calculated after that point in time.

That is, when the second representative value acquisition unit 111 calculates the final representative value Bᵢ at 120 seconds after the start of measurement of the biological information, there is only one already calculated intermediate representative value Aᵢ, and thus, the second representative value acquisition unit 111 also uses only one intermediate representative value Aᵢ to be calculated in the future to calculate the final representative value Bᵢ based on a total of three intermediate representative values Aᵢ including one intermediate representative value Aᵢ at 120 seconds (a lower part (c″) of FIG. 9).

In another example, when the second representative value acquisition unit 111 calculates, for example, the final representative value Bᵢ at 60 seconds from the start of measurement of the biological information, the number of intermediate representative values Aᵢ already calculated is 0 at 60 seconds. Thus, the adjustment unit 112 also adopts 0 for the number of intermediate representative values Aᵢ to be calculated in the future. In this case, the second representative value acquisition unit 111 treats the intermediate representative value Aᵢ at 60 seconds as the final representative value Bᵢ as it is (a lower part (c′) of FIG. 9).

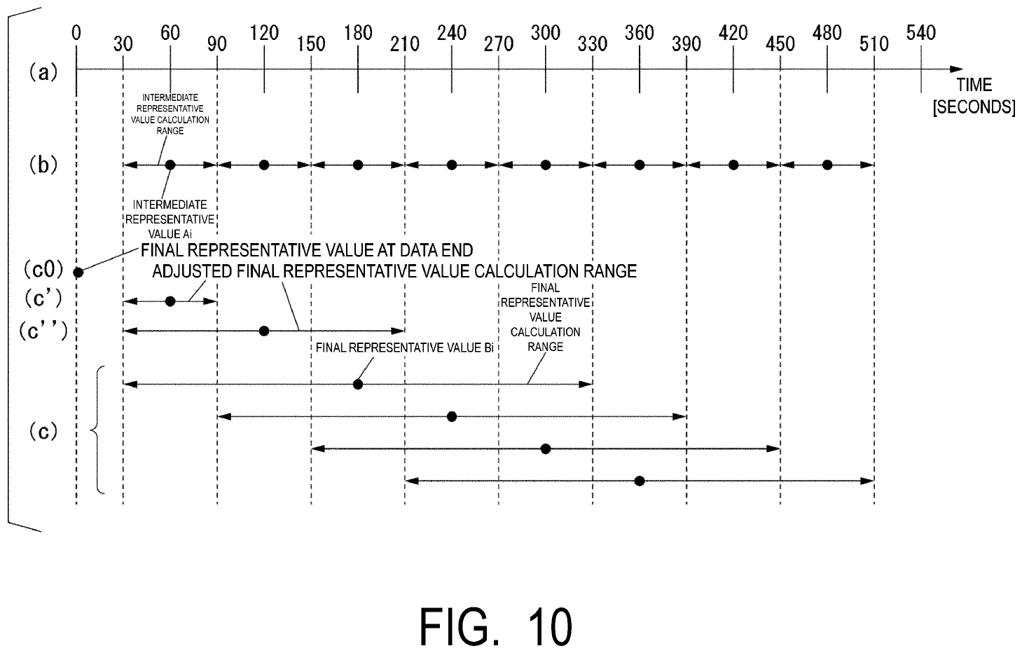

Further, as illustrated in FIG. 10, a measured value of the biological information itself may be complementarily used as the final representative value at a data end (lower part (c0) of FIG. 9).

Operation Sequence of Biological Information Analysis System

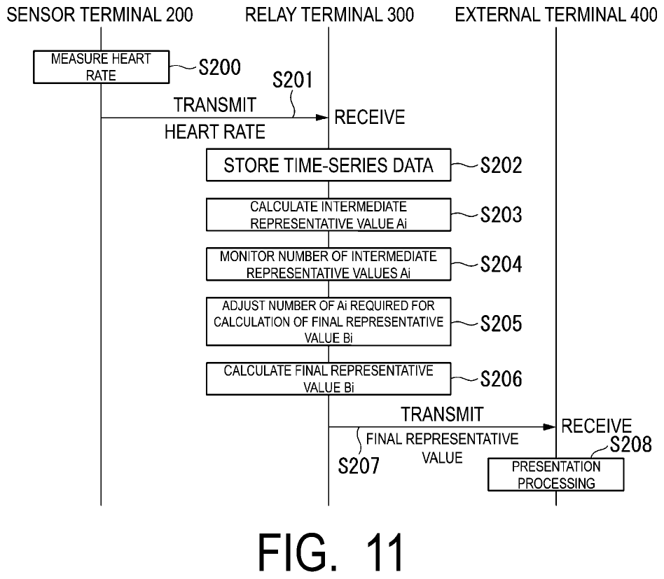

Next, operations that are executed when functions of the biological information analysis apparatus 1A according to the present embodiment are achieved by the biological information analysis system including the sensor terminal 200, the relay terminal 300, and the external terminal 400 described with reference to FIG. 6 will be described with reference to a sequence diagram illustrated in FIG. 11. Respective functional blocks of the sensor terminal 200, the relay terminal 300, and the external terminal 400 are the same as those described in FIG. 6. Further, it is assumed that the relay terminal 300 includes the adjustment unit 112.

First, the sensor terminal 200 is mounted in the user 500 and measures, for example, a heart rate as the biological information of the user 500 (step S200). More specifically, the sensor terminal 200 detects the electrocardiographic potential of the user 500 using a heart rate monitor (the sensor 201). The sensor data acquisition unit 202 acquires the electrocardiographic potential from the sensor 201, and calculates the heart rate from the electrocardiographic waveform based on the electrocardiographic potential. The acquired electrocardiographic potential and heart rate are stored in the data storage unit 203.

Then, the sensor terminal 200 transmits the measured heart rate to the relay terminal 300 via the communication network NW (step S201). More specifically, the data transmission unit 204 reads the time-series data of the heart rate from the data storage unit 203 and transmits the time-series data to the relay terminal 300 via the communication network NW.

When the relay terminal 300 receives the time-series data of the heart rate of the user 500 from the sensor terminal 200, the relay terminal 300 stores the received time-series data in the data storage unit 302 (step S103). The relay terminal 300 calculates the intermediate representative value of the time-series data of the heart rate from the received time-series data in the first representative value acquisition unit 305 in a preset period, for example, every 60 seconds (step S202). The calculated intermediate representative value is stored in the data storage unit 302.

Then, the adjustment unit 112 monitors the number of intermediate representative values calculated by the first representative value acquisition unit 305 (step S203). Thereafter, the adjustment unit 112 determines, for example, the number of intermediate representative values required for calculation of the final representative value, based on the number of intermediate representative values already calculated at a point in time when the second representative value acquisition unit 306 calculates the final representative value according to an external signal or setting (step S204).

Thereafter, the second representative value acquisition unit 306 calculates the final representative value based on the consecutive intermediate representative values corresponding to the number of intermediate representative values determined by the adjustment unit 112 (step S205). Then, the calculated final representative value is transmitted from the relay terminal 300 to the external terminal 400 (step S206).

Thereafter, the external terminal 400 receives the final representative value. The external terminal 400 performs a presentation processing based on the final representative value (step S207) to display the final representative value on the display apparatus or generate and output the support information for the user 500.

### Effects of Second Embodiment

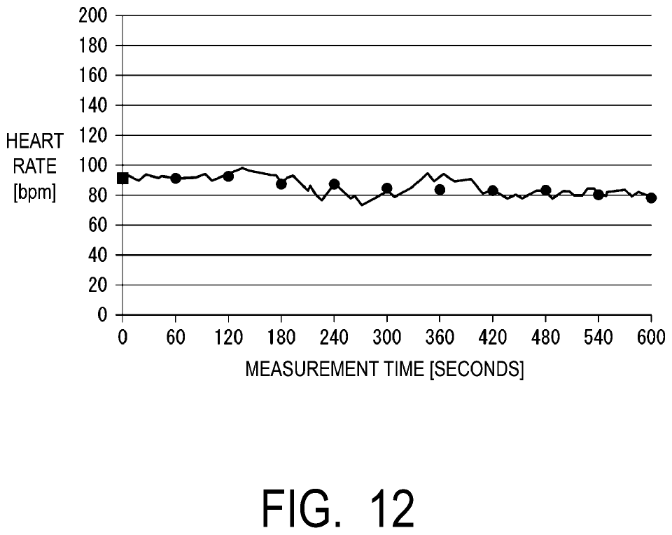

Next, effects of the biological information analysis apparatus 1A according to the present embodiment will be described with reference to FIG. 12. In FIG. 12, a horizontal axis indicates the measurement time (seconds), and a vertical axis indicates the heart rate (bpm). A gray line shown in FIG. 12 indicates the measured heart rate, and circle and square dots indicate the final representative value. For the final representative value at a measurement time of 0 seconds, a value of the measured heart rate is used as it is (square point).

Two circle dots indicating the final representative value of the heart rate at subsequent measurement times of 60 seconds and 120 seconds indicate the final representative value calculated using the number of intermediate representative values determined by the adjustment unit 112. Further, a circle point after the measurement time of 180 seconds indicates the final representative values calculated using five intermediate representative values without performing adjustment of the number of intermediate representative values in the adjustment unit 112.

As illustrated in FIG. 12, it is understood that a measured value of the heart rate (gray line) fluctuates up and down, but the final representative value that moves substantially in a center of the measured value is appropriate as a downsampled moving average.

As described above, with the biological information analysis apparatus 1A according to the second embodiment, the adjustment unit 112 uses the number of already calculated intermediate representative values as the foundation for determining the number of intermediate representative values by which the second representative value acquisition unit 111 calculates the final representative value, and this enables effective utilization of the data near the time of the start or end of the measurement of the biological information and more precise grasp of a behavior of the biological information of the user.

Although, in the second embodiment described above, a case in which the control unit 11A includes the adjustment unit 112 has been described, the adjustment unit 112 may be provided outside the control unit 11A in the biological information analysis apparatus 1A.

### Third Embodiment

Next, a third embodiment of the present invention will be described. In the following description, the same configurations as those of the first and second embodiments described above are denoted by the same reference signs, and description thereof will be omitted.

In the third embodiment, calculation processing of the intermediate representative value differs from those of the first and the second embodiment. In the third embodiment, the processing for calculating the intermediate representative value is performed on the assumption that a communication failure (error) occurs between the sensor terminal 200 and a relay terminal 300B. Hereinafter, a configuration of the third embodiment will be described.

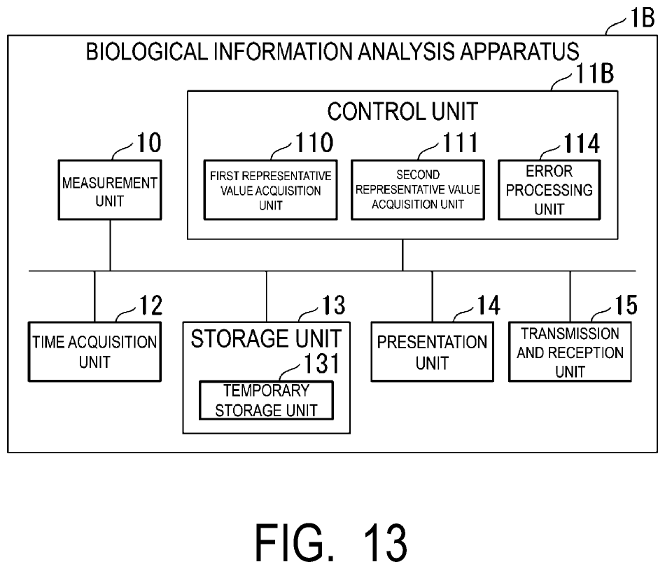

In the third embodiment, a biological information analysis apparatus 1B includes a control unit 11B and a temporary storage unit 131, as illustrated in FIG. 13. The control unit 11B includes a first representative value acquisition unit 110, a second representative value acquisition unit 111, and an error processing unit 114. Other functional configurations included in the biological information analysis apparatus 1B are the same as those in the first embodiment.

The temporary storage unit 131 is set in a part of a storage area of the storage unit 13. When an error occurs in the communication between the transmission and reception unit 15 and the sensor 105, the temporary storage unit 131 temporarily stores the time-series data of the biological information not yet used for calculation of the intermediate representative value at that time.

The error processing unit 114 monitors, based on the number of already calculated intermediate representative values, the number of the pieces of time-series data of the biological information by which the first representative value acquisition unit 110 calculates the intermediate representative value. More specifically, when an error occurs in the communication between the transmission and reception unit 15 and the sensor 105, the error processing unit 114 confirms presence or absence of the time-series data of the biological information not yet used for calculation of the intermediate representative value at that point in time (hereinafter appropriately referred to as pre-error time-series data). When the pre-error time-series data exists, the error processing unit 114 stores the pre-error time-series data in the temporary storage unit 131. The pre-error time-series data is time-series data transmitted before an error occurs.

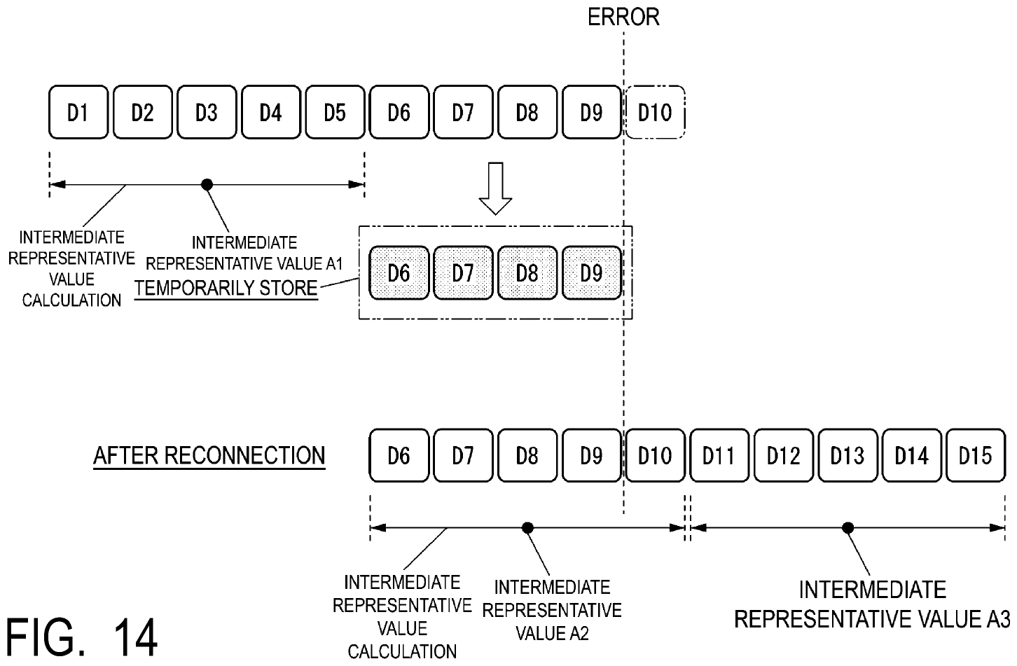

FIG. 14 is a diagram illustrating an example of a data string of time-series data of ecological information transmitted from a sensor. In FIG. 14, each of time-series data D1 to D15 of ecological information indicates time-series data of the ecological information sequentially transmitted from the sensor 105 with the passage of time. In the example of FIG. 4, one intermediate representative value calculation range is set to 60 seconds (t1 (30 seconds) to t2 (90 seconds)). In FIG. 14, one intermediate representative value calculation range is set in the five time-series data (for example, time-series data D1 to D5) of the ecological information. That is, each of the time-series data D1 to D15 in FIG. 14 corresponds to the time-series data of the biological information for 60/5=12 seconds.

As illustrated in FIG. 14, when four time-series data D6 to D9 of the ecological information have not yet been used for calculation of the intermediate representative value at a point in time when the communication error occurs, the error processing unit 114 stores the four time-series data D6 to D9 of the ecological information in the temporary storage unit 131 as the pre-error time-series data.

The error processing unit 114 calls the pre-error time-series data (the time-series data D6 to D9 in the example of FIG. 14) stored in the temporary storage unit 131 when the communication between the transmission and reception unit 15 and the sensor 105 has recovered from the error and the communication between the transmission and reception unit 15 and the sensor 105 is reconnected. The error processing unit 114 uses the called pre-error time-series data and the data transmitted from the sensor 105 after the error occurs (hereinafter appropriately referred to as post-error time-series data) to calculate the intermediate representative value.

More specifically, as illustrated in FIG. 14, the error processing unit 114 uses the four time-series data D6 to D9 of the ecological information called from the temporary storage unit 131 and the time-series data D10 of the ecological information transmitted from the sensor 105 after the error has occurred, to calculate the intermediate representative value.

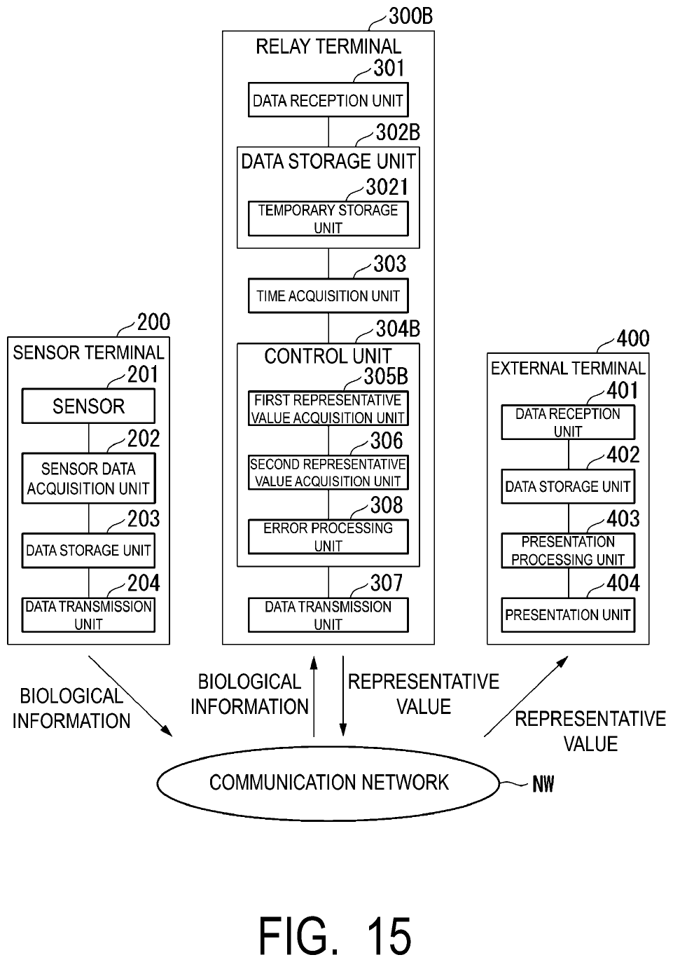

Biological Information Analysis System Next, a biological information analysis system specifically constituting the biological information analysis apparatus 1 according to the present invention will be described with reference to FIG. 15.

The biological information analysis system includes, for example, a sensor terminal 200, the relay terminal 300B, and an external terminal 400, as illustrated in FIG. 15. The sensor terminal 200 and the external terminal 400 have the same configuration as the sensor terminal 200 and the external terminal 400 illustrated in the first embodiment.

Functional Blocks of Relay Terminal

The relay terminal 300B includes a data reception unit 301, a data storage unit 302B, a time acquisition unit 303, a control unit 304B, and a data transmission unit 307.

The data storage unit 302B includes a temporary storage unit 3021. The temporary storage unit 3021 corresponds to the temporary storage unit 131 (FIG. 13).

The control unit 304B includes a first representative value acquisition unit 305B, a second representative value acquisition unit 306, and an error processing unit 308. The error processing unit 308 corresponds to the error processing unit 114 described with reference to FIG. 13.

Operation Sequence of Biological Information Analysis System

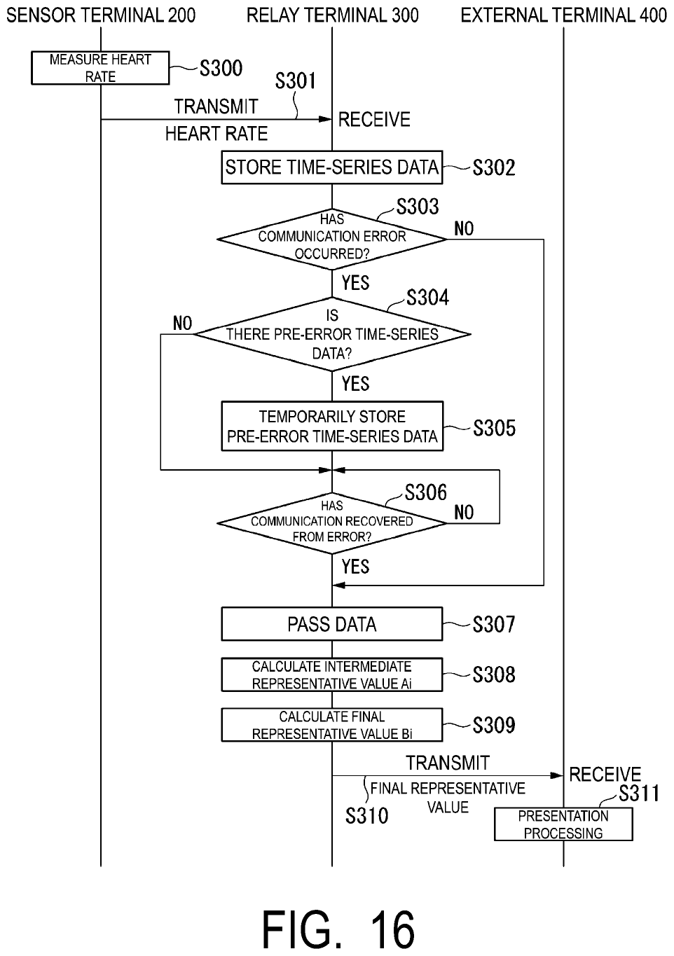

Next, operations that are executed when functions of the biological information analysis apparatus 1B according to the present embodiment are achieved by the biological information analysis system including the sensor terminal 200, the relay terminal 300B, and the external terminal 400 described with reference to FIG. 15 will be described with reference to the sequence diagram illustrated in FIG. 16. Respective functional blocks of the sensor terminal 200, the relay terminal 300B, and the external terminal 400 are the same as those described with reference to FIG. 15.

First, the sensor terminal 200 is mounted in the user 500 and measures, for example, a heart rate as biological information of the user 500 (step S300). More specifically, the sensor terminal 200 detects the electrocardiographic potential of the user 500 using a heart rate monitor (the sensor 201). The sensor data acquisition unit 202 acquires the electrocardiographic potential from the sensor 201, and calculates the heart rate from the electrocardiographic waveform based on the electrocardiographic potential. The acquired electrocardiographic potential and heart rate are stored in the data storage unit 203.

Then, the sensor terminal 200 transmits the measured heart rate to the relay terminal 300B via the communication network NW (step S301). More specifically, the data transmission unit 204 reads the time-series data of the heart rate from the data storage unit 203 and transmits the time-series data to the relay terminal 300B via the communication network NW.

When the relay terminal 300B receives the time-series data of the heart rate of the user 500 from the sensor terminal 200, the relay terminal 300B stores the received time-series data in the data storage unit 302 (step S302).

The error processing unit 308 monitors whether an error has occurred in communication between the sensor terminal 200 and the data reception unit 301 (step S303). When there is no communication error between the sensor terminal 200 and the data reception unit 301, the control unit 304B proceeds to step S308, and the first representative value acquisition unit 305 performs calculation of the intermediate representative value Aᵢ.

When the error processing unit 308 detects in step S303 that an error has occurred in the communication between the sensor terminal 200 and the data reception unit 301, the error processing unit 308 confirms the presence or absence of pre-error time-series data not yet used for calculation of the intermediate representative value at that time (step S304). When the pre-error time-series data does not exist, the error processing unit 308 waits for processing until the communication has recovered from the error. When the pre-error time-series data exists, the error processing unit 308 stores the pre-error time-series data in the temporary storage unit 3021 (step S305).

The error processing unit 308 monitors whether the communication between the sensor terminal 200 and the data reception unit 301 has recovered from the error (step S306). When the communication between the sensor terminal 200 and the data reception unit 301 has recovered from the error, the error processing unit 308 passes the time-series data to the first representative value acquisition unit 305 (step S307). For this purpose, the error processing unit 308 calls the pre-error time-series data from the temporary storage unit 3021. The error processing unit 308 calls, from the data storage unit 302, the time-series data (post-error time-series data) of the ecological information transmitted from the sensor terminal 200 after the error occurs. The error processing unit 308 passes the called pre-error time-series data and post-error time-series data to the first representative value acquisition unit 305.

The first representative value acquisition unit 305 uses the pre-error time-series data and the post-error time-series data to calculate the intermediate representative value Aᵢ every 60 seconds using Equation (2) described above (step S308).

Thereafter, the second representative value acquisition unit 306 calculates the final representative value based on the calculated intermediate representative value (step S309). Then, the calculated final representative value is transmitted from the relay terminal 300B to the external terminal 400 (step S310).

Thereafter, the external terminal 400 receives the final representative value. The external terminal 400 performs a presentation processing based on the final representative value (step S311) to display the final representative value on the display apparatus or generate and output the support information for the user 500.

### Effects of Third Embodiment

As described above, with the biological information analysis apparatus 1B according to the third embodiment, the pre-error time-series data transmitted before the error occurs and stored in the temporary storage unit 3021 and the post-error time-series data transmitted from the sensor terminal 200 after the error occurs are used to acquire the intermediate representative value. This allows to set the time-series data of the ecological information arriving at the relay terminal 300B as a target of processing for calculating the intermediate representative value without omission, and to provide the support information precisely downsampled without any time stamp deviation to the user. As a result, even when a failure occurs in the communication between the sensor terminal 200 and the data reception unit 301, it is possible to curb accuracy degradation and missing of data.

### Fourth Embodiment

Next, a fourth embodiment of the present invention will be described. In the following description, the same configurations as those of the first to third embodiments described above are denoted by the same reference signs, and description thereof will be omitted.

In the fourth embodiment, the processing for calculating the intermediate representative value differs from those in the first to third embodiments. In the fourth embodiment, the processing for calculating the intermediate representative value is performed on the assumption that a communication failure (error) occurs between the sensor terminal 200 and a relay terminal 300C. Hereinafter, a configuration of the fourth embodiment will be described.

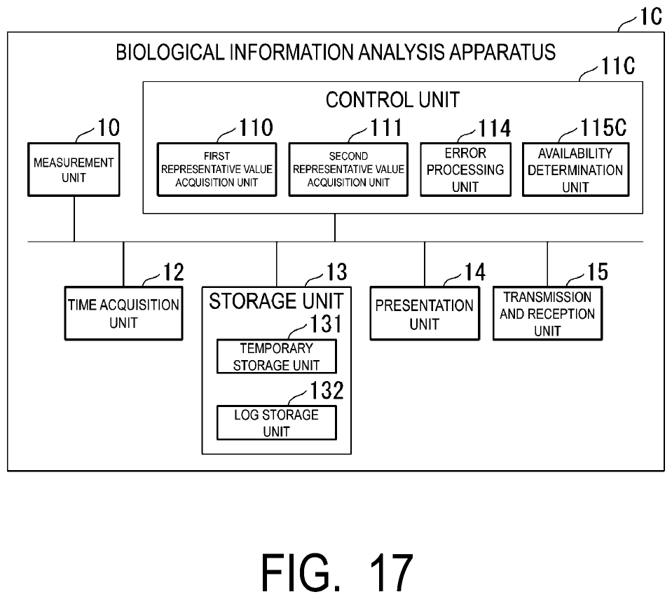

In the fourth embodiment, a biological information analysis apparatus 1C includes a control unit 11C, a temporary storage unit (temporary storage) 131, and a log storage unit (log storage) 132, as illustrated in FIG. 17. The control unit 11C includes a first representative value acquisition unit 110, a second representative value acquisition unit 111, an error processing unit (error processor) 114, and an availability determination unit (availability determinator) 115C. Other functional configurations included in the biological information analysis apparatus 1C are the same as those in the third embodiment.

The log storage unit 132 is set in a part of the storage area of the storage unit 13 together with the temporary storage unit 131. The log storage unit 132 records log information including execution content and an execution time of a procedure in the transmission and reception unit 15 in reception of the time-series data from the sensor 105. The log storage unit 132 stores content of an error when the error occurs in communication with the sensor 105 as the log information together with the time information. The content of the error includes, for example, communication disconnection (disconnect) of Bluetooth Low Energy (BLE), a packet loss, and failure of demodulation. Because the relay terminal 300C performs automatic reconnection of the BLE when the communication disconnection of the BLE has occurred, a log of an automatic reconnection request is also stored as the log information. Further, even when a normal communication ending operation is performed, the record of a procedure thereof is stored as the log information.

Based on the log information stored in the log storage unit 132, the availability determination unit 115C determines whether the pre-error time-series data stored in the temporary storage unit 131 is used for acquisition of the intermediate representative value. More specifically, the availability determination unit 115C refers to log information at the time of communication interruption stored in the log storage unit 132. The availability determination unit 115C determines that the pre-error time-series data can be used for acquisition of the intermediate representative value, for example, when content of the referred log information is related to, for example, a communication failure or automatic reconnection. When the content of the referred log information is similar to the normal communication ending operation, the availability determination unit 115C determines that the pre-error time-series data cannot be used for acquisition of the intermediate representative value.

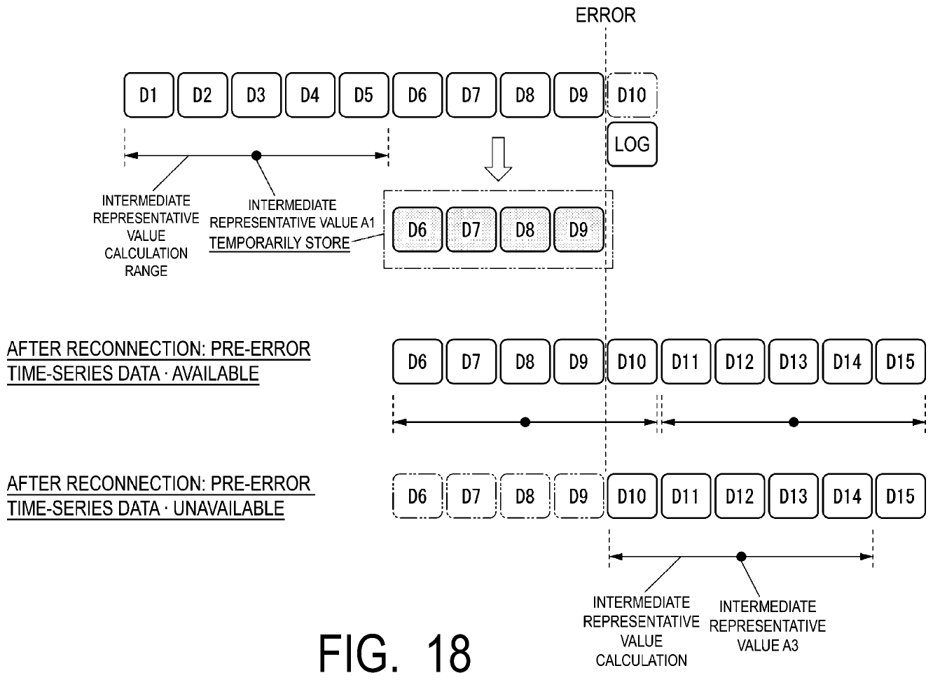

Specifically, for example, as illustrated in FIG. 18, when the four time-series data D6 to D9 of the biological information have not yet been used for calculation of the intermediate representative value at a point in time when a communication error occurs, the availability determination unit 115C stores the four time-series data D6 to D9 of the biological information in the temporary storage unit 131 as the pre-error time-series data.

The availability determination unit 115C determines that the four time-series data D6 to D9 of the biological information stored in the temporary storage unit 131 can be used when the content of the log information recorded when an error has occurred is related to a communication failure or automatic reconnection. The availability determination unit 115C determines that the four time-series data D6 to D9 of the biological information stored in the temporary storage unit 131 cannot be used when the content of the log information is related to the normal communication ending operation.

The error processing unit 114 refers to a determination result of the availability determination unit 115C when the communication between the transmission and reception unit 15 and the sensor 105 has recovered from the error and the communication between the transmission and reception unit 15 and the sensor 105 has been reconnected. When it is determined that the pre-error time-series data stored in the temporary storage unit 131 can be used, the error processing unit 114 calls the time-series data D6 to D9 in a specific example of FIG. 18. The first representative value acquisition unit 110 uses the called pre-error time-series data, and the post-error time-series data (time-series data D10 in the specific example of FIG. 18) transmitted from the sensor 105 after the error occurs, to calculate the intermediate representative value. When it is determined that the pre-error time-series data stored in the temporary storage unit 131 cannot be used, the error processing unit 114 uses only the post-error time-series data (the time-series data D10 to D14 in the specific example of FIG. 18) transmitted from the sensor 105 after the error has occurred, to calculate the intermediate representative value in the specific example of FIG. 18.

Biological Information Analysis System

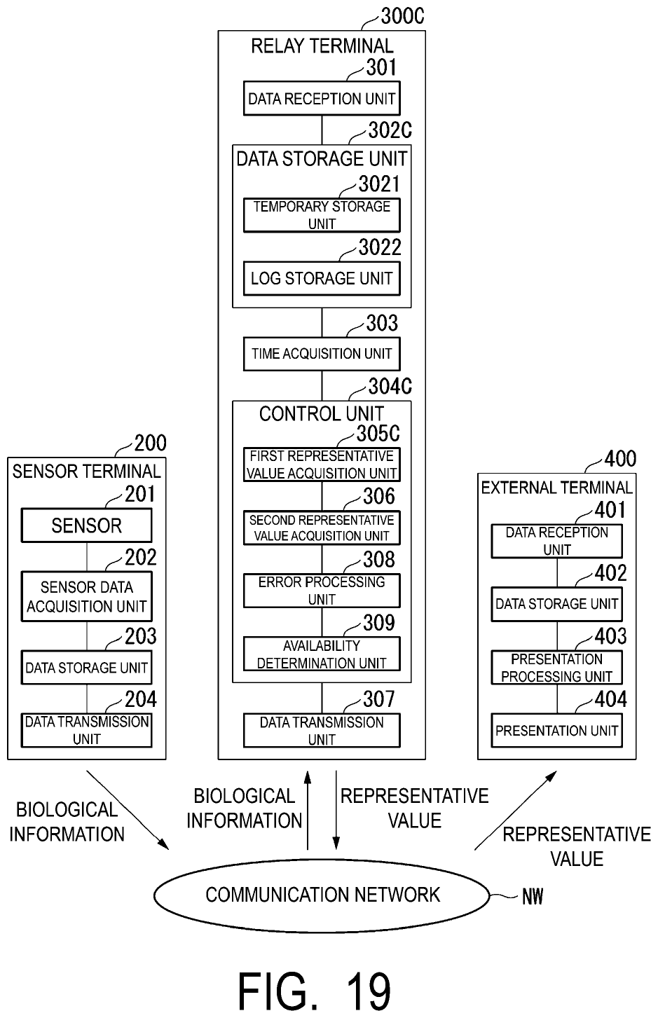

Next, a biological information analysis system specifically constituting the biological information analysis apparatus 1 according to the present invention will be described with reference to FIG. 19.

The biological information analysis system includes, for example, a sensor terminal 200, the relay terminal 300C, and an external terminal 400, as illustrated in FIG. 19. The sensor terminal 200 and the external terminal 400 have the same configuration as the sensor terminal 200 and the external terminal 400 illustrated in the first embodiment.

Functional Blocks of Relay Terminal

The relay terminal 300C includes a data reception unit 301, a data storage unit 302C, a time acquisition unit 303, a control unit 304C, and a data transmission unit 307.

The data storage unit 302C includes a temporary storage unit 3021 and a log storage unit 3022. The log storage unit 3022 corresponds to the log storage unit 132 (FIG. 17).

The control unit 304C includes a first representative value acquisition unit (first representative value acquisitor) 305C, a second representative value acquisition unit (second representative value acquisitor) 306, an error processing unit (error processor) 308, and an availability determination unit (availability determinator) 309. The availability determination unit 309 corresponds to the availability determination unit 115C described with reference to FIG. 17.

Operation Sequence of Biological Information Analysis System

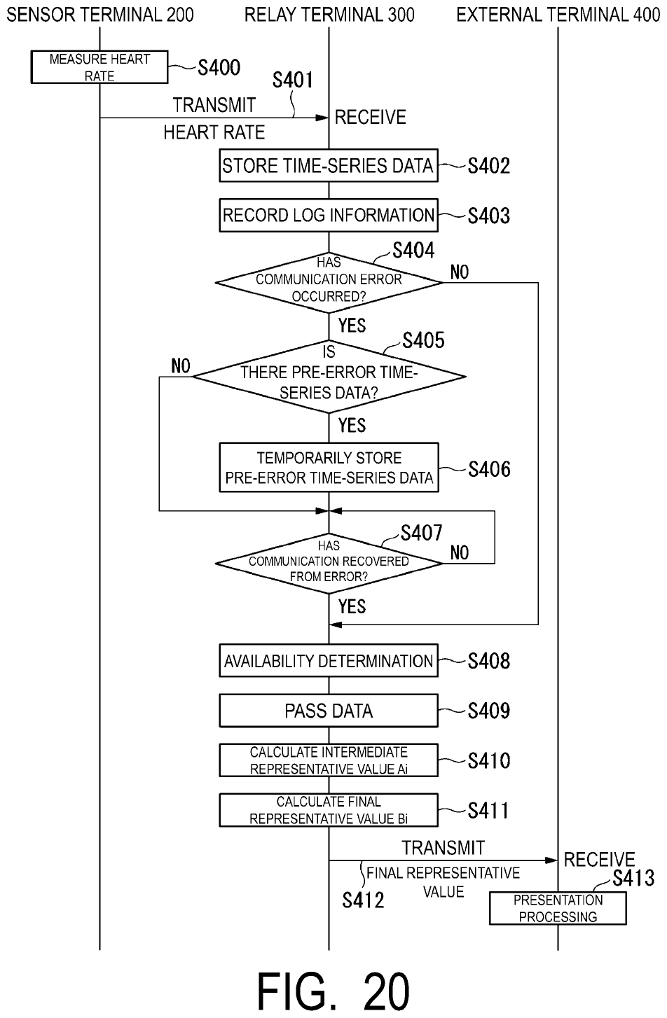

Next, operations that are executed when functions of the biological information analysis apparatus 1C according to the present embodiment are achieved by the biological information analysis system including the sensor terminal 200, the relay terminal 300C, and the external terminal 400 described with reference to FIG. 19 will be described with reference to the sequence diagram illustrated in FIG. 20. Respective functional blocks of the sensor terminal 200, the relay terminal 300C, and the external terminal 400 are the same as those described in FIG. 19.

First, the sensor terminal 200 is mounted in the user 500, and measures, for example, the heart rate as the biological information of the user 500 (step S400). More specifically, the sensor terminal 200 detects the electrocardiographic potential of the user 500 using a heart rate monitor (the sensor 201). The sensor data acquisition unit 202 acquires the electrocardiographic potential from the sensor 201, and calculates the heart rate from the electrocardiographic waveform based on the electrocardiographic potential. The acquired electrocardiographic potential and heart rate are stored in the data storage unit 203.

Then, the sensor terminal 200 transmits the measured heart rate to the relay terminal 300C via the communication network NW (step S401). More specifically, the data transmission unit 204 reads the time-series data of the heart rate from the data storage unit 203 and transmits the time-series data to the relay terminal 300C via the communication network NW.

When the relay terminal 300C receives the time-series data of the heart rate of the user 500 from the sensor terminal 200, the relay terminal 300C stores the received time-series data in the data storage unit 302 (step S402).

When the log storage unit 132 receives the time-series data of the heart rate from the sensor terminal 200, the log storage unit 132 records the log information including execution content and an execution time of a procedure in the data reception unit 301 in reception of the time-series data from the sensor terminal 200 (step S403).

The error processing unit 308 monitors whether an error has occurred in the communication between the sensor terminal 200 and the data reception unit 301 (step S404). When there is no communication error between the sensor terminal 200 and the data reception unit 301, the control unit 304C proceeds to step S410 and the first representative value acquisition unit 305 performs calculation of the intermediate representative value Aᵢ.

When it is detected in step S404 that an error has occurred in the communication between the sensor terminal 200 and the data reception unit 301, the error processing unit 308 confirms the presence or absence of the pre-error time-series data not yet used for calculation of the intermediate representative value (step S405). When the pre-error time-series data does not exist, the error processing unit 308 waits for processing until the communication has recovered from the error. When the pre-error time-series data exists, the error processing unit 308 stores the pre-error time-series data in the temporary storage unit 3021 (step S406).

The error processing unit 308 monitors whether the communication between the sensor terminal 200 and the data reception unit 301 has recovered from the error (step S407). When the communication between the sensor terminal 200 and the data reception unit 301 has recovered from the error, the availability determination unit 309 determines whether the pre-error time-series data can be used (step S408). Based on the log information, the availability determination unit 309 determines whether the pre-error time-series data stored in the temporary storage unit 3021 is used for acquisition of the intermediate representative value. More specifically, the availability determination unit 309 refers to the log information at the time of communication interruption stored in the log storage unit 3022. The availability determination unit 309 determines that the pre-error time-series data can be used for acquisition of the intermediate representative value, when content of the referred log information is related to, for example, a communication failure or automatic reconnection. The availability determination unit 309 determines that the pre-error time-series data cannot be used for acquisition of the intermediate representative value when the content of the referred log information is similar to the normal communication ending operation.

The error processing unit 308 passes the time-series data to the first representative value acquisition unit 305 based on a determination result of the availability determination unit 309 (step S409). When the availability determination unit 309 determines that the pre-error time-series data can be used for acquisition of the intermediate representative value, the error processing unit 308 calls the pre-error time-series data from the temporary storage unit 3021. The error processing unit 308 calls, from the data storage unit 302, the time-series data (post-error time-series data) of the biological information transmitted from the sensor terminal 200 after the error occurs. The error processing unit 308 passes the called pre-error time-series data and post-error time-series data to the first representative value acquisition unit 305. Further, when the availability determination unit 309 determines that the pre-error time-series data cannot be used for acquisition of the intermediate representative value, the error processing unit 308 calls, from the data storage unit 302, the time-series data (post-error time-series data) of the biological information transmitted from the sensor terminal 200 after the error occurs. The error processing unit 308 passes the post-error time-series data to the first representative value acquisition unit 305.

When it is determined that the pre-error time-series data can be used for acquisition of the intermediate representative value, the first representative value acquisition unit 305 uses the pre-error time-series data and the post-error time-series data to calculate the intermediate representative value Aᵢ (step S410). When a determination is made that the pre-error time-series data cannot be used for acquisition of the intermediate representative value, the first representative value acquisition unit 305 uses only the post-error time-series data to calculate the intermediate representative value Aᵢ.

Thereafter, the second representative value acquisition unit 306 calculates the final representative value based on the calculated intermediate representative value (step S411). Then, the calculated final representative value is transmitted from the relay terminal 300C to the external terminal 400 (step S412).

Thereafter, the external terminal 400 receives the final representative value. The external terminal 400 performs a presentation processing based on the final representative value (step S413) to display the final representative value on the display apparatus or generate and output the support information for the user 500.

### Effects of Fourth Embodiment

As described above, with the biological information analysis apparatus 1C according to the fourth embodiment, the pre-error time-series data transmitted before the error occurs and stored in the temporary storage unit 3021 and the post-error time-series data transmitted from the sensor terminal 200 after the error occurs are used to acquire the intermediate representative value. Further, based on the log information, it is determined whether the pre-error time-series data can be used according to a situation when the communication error has occurred. Thus, for example, when the user stops the continuation of communication, it is possible to stop the calculation processing and, in other cases, to set the time-series data of the biological information arriving at the relay terminal 300C as a target of processing for calculating the intermediate representative value without omission. As a result, even when a failure occurs in the communication between the sensor terminal 200 and the data reception unit 301, it is possible to curb accuracy degradation and missing of data.

### Fifth Embodiment

Next, a fifth embodiment of the present invention will be described. In the following description, the same configurations as those of the first to fourth embodiments described above are denoted by the same reference signs, and description thereof will be omitted.

In the fifth embodiment, the processing for calculating the intermediate representative value differs from those in the first to fourth embodiments. In the fifth embodiment, the processing for calculating the intermediate representative value is performed on the assumption that a communication failure (error) occurs between the sensor terminal 200 and a relay terminal 300D. Hereinafter, a configuration of the fifth embodiment will be described.

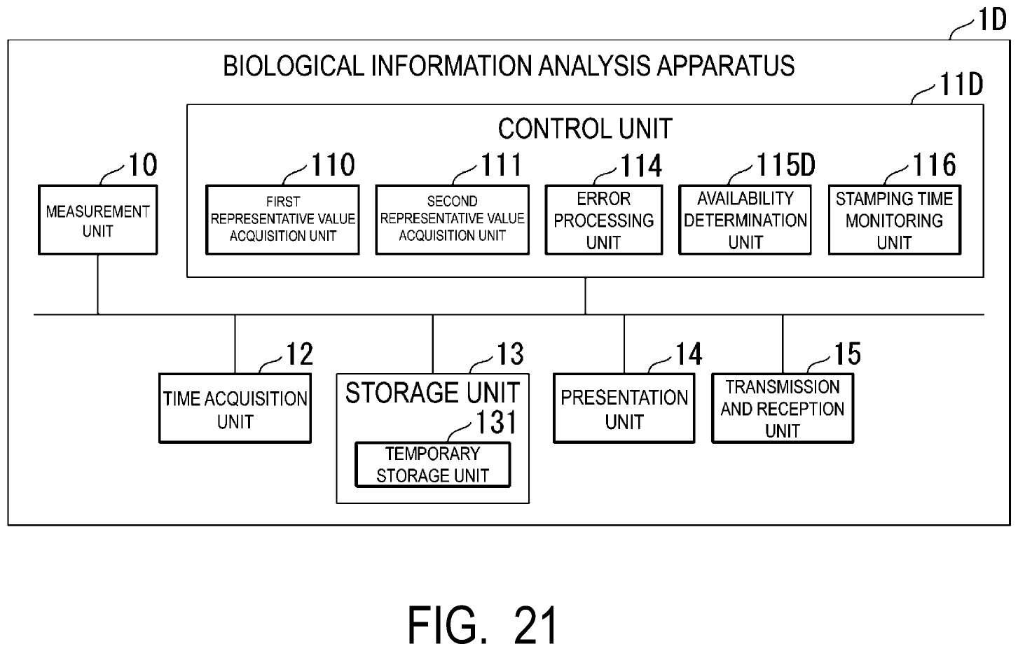

In the fifth embodiment, a biological information analysis apparatus 1D includes a control unit 11D and a temporary storage unit 131, as illustrated in FIG. 21. The control unit 11D includes a first representative value acquisition unit 110, a second representative value acquisition unit 111, an error processing unit 114, an availability determination unit 115D, and a stamping time monitoring unit (stamping time monitor) 116. Other functional configurations included in the biological information analysis apparatus 1D are the same as those in the third embodiment.

In the present embodiment, the sensor 105 transmits information indicating a time when the time-series data has been acquired, in association with the time-series data. Further, in the present embodiment, information indicating a time when the time-series data transmitted by the sensor 105 arrives at the transmission and reception unit 15 may be associated with the time-series data.

The stamping time monitoring unit 116 monitors the time when the time-series data has been acquired by the sensor 105 or a time when the time-series data has arrived at the transmission and reception unit 15. When an error occurs in communication with the sensor 105, the stamping time monitoring unit 116 determines whether a time interval between the time information of the pre-error time-series data and the time information of the post-error time-series data exceeds a predetermined threshold value.

The availability determination unit 115D determines whether the pre-error time-series data stored in the temporary storage unit 131 is used for acquisition of the intermediate representative value, based on a determination result for monitoring in the stamping time monitoring unit 116. More specifically, the availability determination unit 115D determines that the pre-error time-series data can be used for acquisition of the intermediate representative value when the time interval between the time information of the pre-error time-series data and the time information of the post-error time-series data does not exceed a predetermined threshold value. When the time interval between the time information of the pre-error time-series data and the time information of the post-error time-series data exceeds the predetermined threshold value, the availability determination unit 115D determines that the pre-error time-series data cannot be used for acquisition of the intermediate representative value.

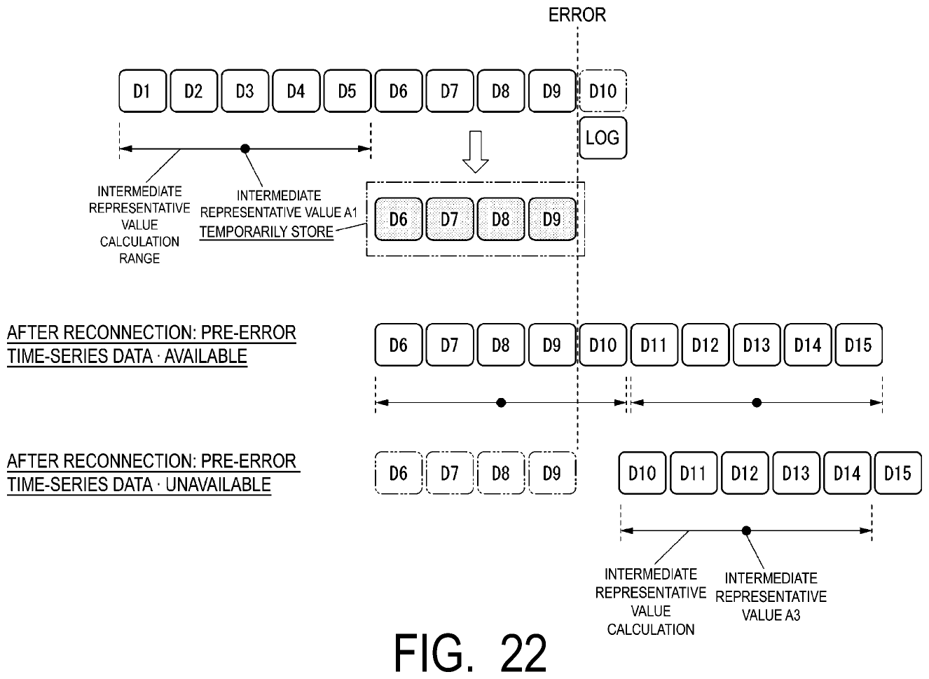

Specifically, for example, as illustrated in FIG. 22, when the four time-series data D6 to D9 of the biological information have not yet been used for calculation of the intermediate representative value at a point in time when a communication error occurs, the availability determination unit 115D stores the four time-series data D6 to D9 of the biological information in the temporary storage unit 131 as the pre-error time-series data.

The availability determination unit 115D determines that the pre-error time-series data can be used when the time interval between the time information of the pre-error time-series data and the time information of the post-error time-series data does not exceed the predetermined threshold value before and after the error occurs. The availability determination unit 115D determines that the four time-series data D6 to D9 of the biological information stored in the temporary storage unit 131 cannot be used when the time interval between the time information of the pre-error time-series data and the time information of the post-error time-series data exceeds the predetermined threshold value.

The error processing unit 114 refers to a determination result of the availability determination unit 115D when the communication between the transmission and reception unit 15 and the sensor 105 has recovered from the error and the communication between the transmission and reception unit 15 and the sensor 105 has been reconnected. When it is determined that the pre-error time-series data stored in the temporary storage unit 131 can be used, the error processing unit 114 calls the time-series data D6 to D9 in a specific example of FIG. 22. The first representative value acquisition unit 110 uses the called pre-error time-series data, and the post-error time-series data (time-series data D10 in the specific example of FIG. 22) transmitted from the sensor 105 after the error occurs, to calculate the intermediate representative value. When it is determined that the pre-error time-series data stored in the temporary storage unit 131 cannot be used, the error processing unit 114 uses only the post-error time-series data (time-series data D10 to D14 in the specific example of FIG. 22) transmitted from the sensor 105 after the error has occurred, to calculate the intermediate representative value in the specific example of FIG. 22.

Biological Information Analysis System

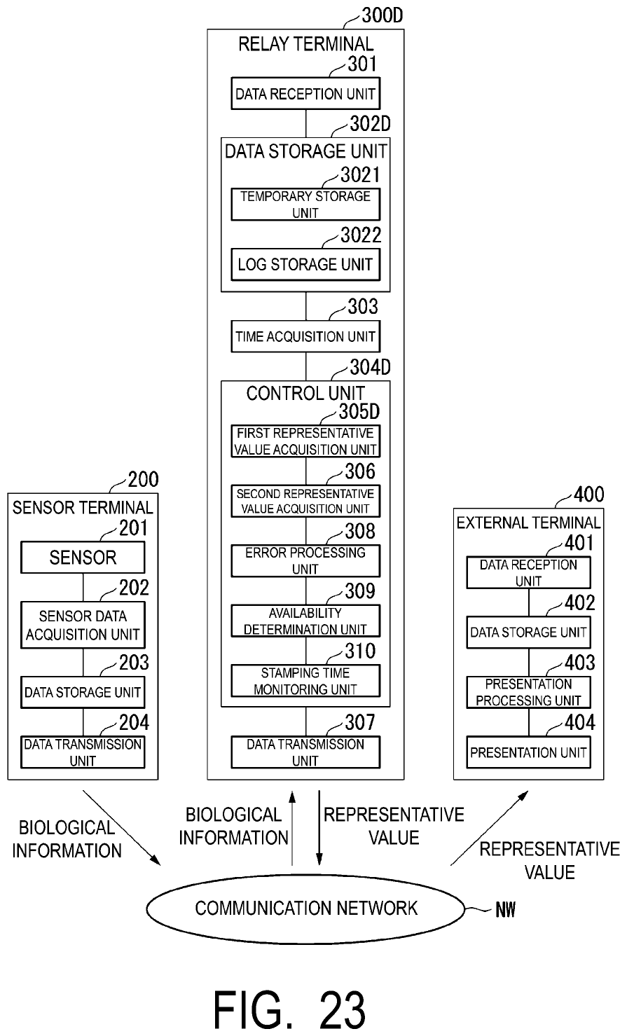

Next, a biological information analysis system specifically constituting the biological information analysis apparatus 1 according to the present invention will be described with reference to FIG. 23.

As illustrated in FIG. 23, the biological information analysis system includes, for example, a sensor terminal 200, the relay terminal 300D, and an external terminal 400. The sensor terminal 200 and the external terminal 400 have the same configuration as the sensor terminal 200 and the external terminal 400 illustrated in the first embodiment.

Functional Blocks of Relay Terminal

The relay terminal 300D includes a data reception unit 301, a data storage unit 302D, a time acquisition unit 303, a control unit 304D, and a data transmission unit 307.

The control unit 304D includes a first representative value acquisition unit 305D, a second representative value acquisition unit 306, an error processing unit 308, an availability determination unit 309, and a stamping time monitoring unit (stamping time monitor) 310. The stamping time monitoring unit 310 corresponds to the stamping time monitoring unit 116 described with reference to FIG. 21.

Operation Sequence of Biological Information Analysis System

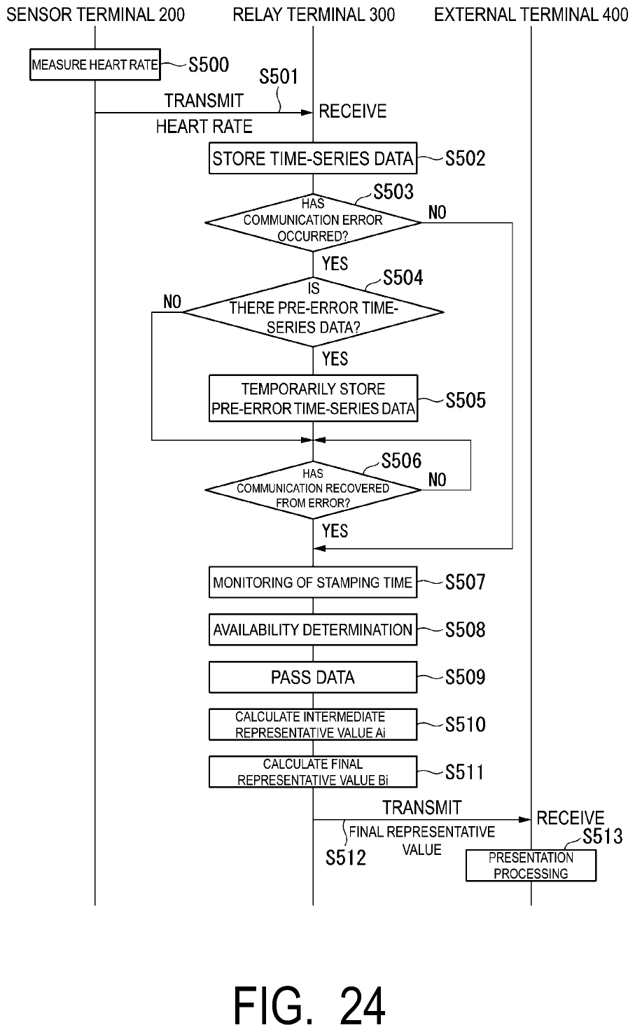

Next, operations that are executed when functions of the biological information analysis apparatus 1D according to the present embodiment are achieved by the biological information analysis system including the sensor terminal 200, the relay terminal 300D, and the external terminal 400 described with reference to FIG. 23 will be described with reference to a sequence diagram illustrated in FIG. 24. Respective functional blocks of the sensor terminal 200, the relay terminal 300D, and the external terminal 400 are the same as those described with reference to FIG. 23.

First, the sensor terminal 200 is mounted in the user 500 and measures, for example, the heart rate as the biological information of the user 500 (step S500). More specifically, the sensor terminal 200 detects the electrocardiographic potential of the user 500 using a heart rate monitor (the sensor 201). The sensor data acquisition unit 202 acquires the electrocardiographic potential from the sensor 201, and calculates the heart rate from the electrocardiographic waveform based on the electrocardiographic potential. The acquired electrocardiographic potential and heart rate are stored in the data storage unit 203. The sensor data acquisition unit 202 stores information on a time when the sensor 201 has acquired the electrocardiographic potential, in association with the electrocardiographic potential.

Then, the sensor terminal 200 transmits the measured heart rate to the relay terminal 300D via the communication network NW (step S501). More specifically, the data transmission unit 204 reads the time-series data of the heart rate from the data storage unit 203 and transmits the time-series data to the relay terminal 300D via the communication network NW.

When the relay terminal 300D receives the time-series data of the heart rate of the user 500 from the sensor terminal 200, the relay terminal 300D stores the received time-series data in the data storage unit 302 (step S502).

When the time-series data of the heart rate is received from the sensor terminal 200, the stamping time monitoring unit 116 refers to the time information associated with the time-series data (step S503).

The error processing unit 308 monitors whether an error has occurred in the communication between the sensor terminal 200 and the data reception unit 301 (step S503). When there is no communication error between the sensor terminal 200 and the data reception unit 301, the control unit 304D proceeds to step S510 and the first representative value acquisition unit 305 calculates the intermediate representative value Aᵢ.

When it is detected in step S503 that an error has occurred in the communication between the sensor terminal 200 and the data reception unit 301, the error processing unit 308 confirms the presence or absence of the pre-error time-series data not yet used for calculation of the intermediate representative value (step S504). When the pre-error time-series data does not exist, the error processing unit 308 waits for processing until the communication has recovered from the error. When the pre-error time-series data exists, the error processing unit 308 stores the pre-error time-series data in the temporary storage unit 3021 (step S505).

The error processing unit 308 monitors whether the communication between the sensor terminal 200 and the data reception unit 301 has recovered from the error (step S506). When the communication between the sensor terminal 200 and the data reception unit 301 has recovered from the error, the stamping time monitoring unit 310 monitors time information of time-series information that has arrived before and after the error (step S507). The stamping time monitoring unit 310 determines whether the time interval between the time information of the pre-error time-series data and the time information of the post-error time-series data exceeds the predetermined threshold value.

Subsequently, based on a determination result for monitoring in the stamping time monitoring unit 310, the availability determination unit 309 determines whether the pre-error time-series data stored in the temporary storage unit 3021 is used for acquisition of the intermediate representative value (step S50). The availability determination unit 115D determines that the pre-error time-series data can be used for acquisition of the intermediate representative value when the time interval between the time information of the pre-error time-series data and the time information of the post-error time-series data does not exceed the predetermined threshold value. The availability determination unit 309 determines that the pre-error time-series data cannot be used for acquisition of the intermediates representative value when the time interval between the time information of the pre-error time-series data and the time information of the post-error time-series data exceeds the predetermined threshold value. The error processing unit 308 passes the time-series data to the first representative value acquisition unit 305 based on a determination result of the availability determination unit 309 (step S509). When the availability determination unit 309 determines that the pre-error time-series data can be used for acquisition of the intermediate representative value, the error processing unit 308 calls the pre-error time-series data from the temporary storage unit 3021. The error processing unit 308 calls, from the data storage unit 302, the time-series data (post-error time-series data) of the biological information transmitted from the sensor terminal 200 after the error occurs. The error processing unit 308 passes the called pre-error time-series data and post-error time-series data to the first representative value acquisition unit 305. Further, when the availability determination unit 309 determines that the pre-error time-series data cannot be used for acquisition of the intermediate representative value, the error processing unit 308 calls, from the data storage unit 302, the time-series data (post-error time-series data) of the biological information transmitted from the sensor terminal 200 after the error occurs. The error processing unit 308 passes the post-error time-series data to the first representative value acquisition unit 305.

When a determination is made that the pre-error time-series data can be used for acquisition of the intermediate representative value, the first representative value acquisition unit 305 uses the pre-error time-series data and the post-error time-series data to calculate the intermediate representative value Aᵢ (step S510). When a determination is made that the pre-error time-series data cannot be used for acquisition of the intermediate representative value, the first representative value acquisition unit 305 uses only the post-error time-series data to calculate the intermediate representative value Aᵢ.

Thereafter, the second representative value acquisition unit 306 calculates the final representative value based on the calculated intermediate representative value (step S511). Next, the calculated final representative value is transmitted from the relay terminal 300D to the external terminal 400 (step S512).

Thereafter, the external terminal 400 receives the final representative value. The external terminal 400 performs a presentation processing based on the final representative value (step S513) to display the final representative value on the display apparatus or generate and output the support information for the user 500.

### Effects of Fifth Embodiment

As described above, with the biological information analysis apparatus 1D according to the fifth embodiment, the pre-error time-series data transmitted before the error occurs and stored in the temporary storage unit 3021 and the post-error time-series data transmitted from the sensor terminal 200 after the error occurs are used to acquire the intermediate representative value. Further, the availability of the pre-error time-series data is determined based on a time interval of the time-series data before and after the error. Thus, for example, when a stop time due to an error is long, the calculation processing using the pre-error time-series data is stopped and, in other cases, the time-series data of the biological information arriving at the relay terminal 300D can be set as a target of processing for calculating the intermediate representative value without omission. As a result, even when a failure occurs in the communication between the sensor terminal 200 and the data reception unit 301, it is possible to curb accuracy degradation and missing of data.

### Modification Examples of Fourth and Fifth Embodiments

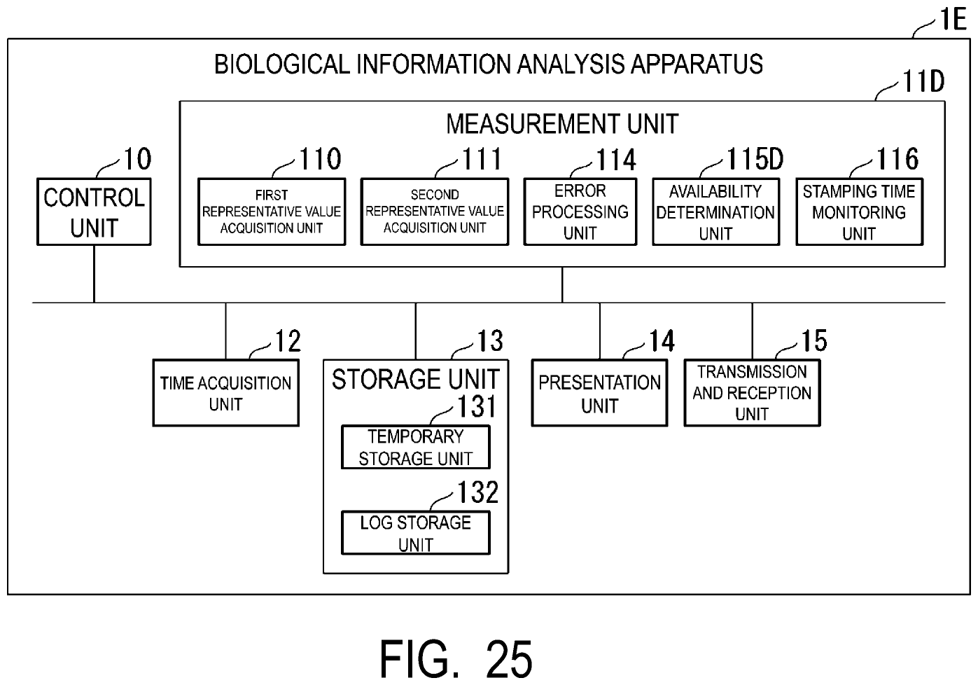

The biological information analysis system according to the present modification example is a modification example of the fourth embodiment and the fifth embodiment. With such a modification example, as illustrated in FIG. 25, an ecological information analysis apparatus 1E in the present embodiment includes the log storage unit 132 described in the fourth embodiment and the stamping time monitoring unit 116 described in the fifth embodiment.

In such a configuration, even when the content of the log information is related to communication failure or automatic reconnection, the pre-error time-series data is not used in calculation processing as long as a time interval of the time information of the time-series data exceeds a certain threshold value. Thus, for example, when a situation of the user is highly likely to change due to the passage of a long time, it is possible to perform processing for calculating the representative value using only the post-error time-series data without using the pre-error time-series data. Thus, it is possible to appropriately ascertain, from a communication situation, whether an operation intended by the user has been performed, to perform processing for calculating the representative value according to a situation of the user from a stamping interval, and to provide a more reliable downsampled average value.

### Sixth Embodiment

Next, a sixth embodiment of the present invention will be described. In the following description, the same configurations as those in the first to fifth embodiments described above are denoted by the same reference signs, and description thereof will be omitted.

In the first to fifth embodiments, a case in which the average value is calculated as the intermediate representative value and the final representative value of the time-series data of the biological information has been illustrated and described. On the other hand, in the sixth embodiment, a proportion is obtained instead of the average value, as the statistical representative value of the time-series data of the biological information.

The configuration of the biological information analysis apparatus 1 according to the present embodiment is the same as the configuration of the biological information analysis apparatus 1 illustrated in FIG. 1. Further, the configuration of the biological information analysis system according to the present embodiment is the same as the configuration illustrated in FIG. 6. Hereinafter, configurations different from those of the first to fifth embodiments will be mainly described.

For the heart rate, which is one of the biological information, a body temperature, a blood pressure, and the like, an average can be calculated in the time series illustrated in the first embodiment. However, calculation of an average may not be appropriate for other biological information. An example thereof may include a case in which a state of the user has been estimated by a sensor. When a state in which the user is lying (sleeping) is 0, a state in which the user is awake is 1, and the state of the user is always classified into either of these two values, an average of these values is 0.5, which does not make sense.

Information that can take an intermediate value such as a heart rate is called a quantitative variable, and information that does not allow an intermediate value indicating a state such as a posture is called a qualitative variable. In the biological information analysis apparatus 1, when such a qualitative variable is analyzed, it is preferable to use a proportion indicating a frequency at which the qualitative variable is generated.

For example, it is assumed that a lying state (sleeping) is 0, an awake state is 1, and a walking state is 2, and the intermediate representative value Aᵢ is obtained every 60 seconds at a sampling rate of one second. In this case, in a case in which a lying period is 30 seconds, an awake period is 20 seconds, and a walking period is 10 seconds, and these numbers are N0,i, N1,i, and N2,i, the intermediate representative value Aᵢ is expressed by Equation (3) below.

Aᵢ=(N_{0,1}N_{1,1}N_{2,i})=(302010) [Math. 3]

Further, in the case of Equation (3), for example, when a most frequent value is taken as the final representative value Bᵢ, the final representative value Bᵢ is calculated using Equation (4) below.

$$\begin{matrix}[ Math . 4 ] & \\ {B}_{i} = MAX ( {{\sum }_{k = - 2}}^{K = + 2} {N}_{0 , i + k} {{\sum }_{k = - 2}}^{K = + 2} {N}_{1 , i + k} {{\sum }^{K = + 2}}_{k = - 2} {N}_{2 , i + k} ) = 1 & ( 4 )\end{matrix}$$

An output, the final representative value Bᵢ=1, in Equation (4) above is an example, and indicates, for example, content that an awake state was most frequent. However, a configuration in which the most frequent value is taken as the final representative value Bᵢ is an example, and another determination method may be used. For example, in the three states including a lying state, an awake state, and a walking state described above, walking is considered to be difficult to occur because the walking uses the most physical strength. Thus, for example, when the user walks for 6 seconds or more, the walking state may be preferentially selected.

In this case, the intermediate representative value Aᵢ is expressed by Equation (5) below.

Aᵢ=(N_{0,i}N_{1,i}N_{2,i})=(302010)=2 [Math. 5]

In a case in which a state in which the user is walking is output as the intermediate representative value Ai by using Equation (5) above, for example, when the user does not walk for six seconds or more, a state (0 or 1) of the longer of the lying period N0,i and the awake period N1,i is obtained by majority and is used as the intermediate representative value Aᵢ.

When the final representative value Bi is calculated based on the intermediate representative value Ai calculated using Equation (5) above, calculation for determining the final representative value Bi through a determination that the state is regarded as walking when the intermediate representative value Ai indicating the walking state of the user among a plurality of consecutive intermediate representative values Ai appears once or more is performed.

However, because a numerical value indicating a state such as whether the user has walked is very rapid as compared with an increase or decrease of a value such as a heart rate or blood pressure, the intermediate representative value Ai may be equal to the final representative value Bi as it is. In particular, when a plurality of sensors are used together, the quantitative variable is more appropriate for intuition of the user when the final representative value Bi is calculated through calculation based on the intermediate representative value Ai, and the qualitative variable is more appropriate for intuition of the user when the intermediate representative value Ai is used as the final representative value Bi as it is.

Operation Sequence of Biological Information Analysis System Next, operations that are executed when functions of the biological information analysis apparatus 1 according to the present embodiment are achieved by the biological information analysis system including the sensor terminal 200, the relay terminal 300, and the external terminal 400 described with reference to FIG. 6 will be described with reference to a sequence diagram illustrated in FIG. 17. Respective functional blocks of the sensor terminal 200, the relay terminal 300, and the external terminal 400 are the same as those described in FIG. 6.

First, the sensor terminal 200 is mounted in the user 500, and measures, for example, a posture and walking as the biological information of the user 500 (step S700). More specifically, the sensor terminal 200 detects acceleration data of the user 500 using a three-axis acceleration sensor (the sensor 201). The sensor data acquisition unit 202 acquires the acceleration data from the sensor 201, and measures a lying state, an awake state, and a walking state of the user 500 from inclination or body movement based on the acceleration data. The measured time-series data of the biological information indicating the posture and walking state of the user is stored in the data storage unit 203.

Then, the sensor terminal 200 transmits the measured biological information indicating the posture and walking of the user to the relay terminal 300 via the communication network NW (step S701). More specifically, the data transmission unit 204 reads the time-series data of the biological information indicating the state of the user from the data storage unit 203 and transmits the time-series data to the relay terminal 300 via the communication network NW.

When the relay terminal 300 receives the time-series data of the biological information indicating the state of the user 500 from the sensor terminal 200, the relay terminal 300 calculates the intermediate representative value in the time-series data of the state of the user 500 for a preset period, such as every 60 seconds in the first representative value acquisition unit 305 (step S702). More specifically, the first representative value acquisition unit 305 uses, for example, Equation (4) described above to calculate a proportion of periods in which a lying state, an awake state, and a walking state of the user 500 occur, as an intermediate representative value every 60 seconds. The calculated intermediate representative value is stored in the data storage unit 302.

Thereafter, the second representative value acquisition unit 306 calculates the final representative value based on the calculated intermediate representative value (step S703). More specifically, the second representative value acquisition unit 306 may calculate the final representative value with a highest frequency using Equation (5) above. Then, the calculated final representative value is transmitted from the relay terminal 300 to the external terminal 400 (step S704).

Thereafter, the external terminal 400 receives the final representative value. The external terminal 400 performs a presentation processing based on the final representative value (step S705) to display the final representative value on the display apparatus or generate and output support information for the user 500.

As described above, with the biological information analysis apparatus 1 according to the sixth embodiment, use of a proportion of any period as the intermediate representative value of the biological information also allows application to a sensor that measures the biological information corresponding to the qualitative variable.

Although the embodiments of the biological information analysis system, the non-transitory computer readable medium, and the biological information analysis method of the present invention have been described above, the present invention is not limited to the described embodiments and it is possible to make various modifications that can be assumed by those skilled in the art within the scope of the invention described in the claims.

In the described embodiments, a case in which the heart rate, acceleration, posture and walking are used as the biological information measured by the sensor data acquisition unit 10 has been described, but the biological information is not limited thereto and may be, for example, a pulse wave, blood pressure, biological impedance, myoelectric potential, respiration, moving speed, angular velocity, an atmospheric pressure, light, and temperature and humidity.

### INDUSTRIAL APPLICABILITY

Provided is a technology capable of curbing accuracy degradation and missing of data even when a failure occurs in data communication.

### REFERENCE SIGNS LIST

1, 1A, 1B, 1C, 1D Biological information analysis apparatus
11, 11A, 11B, 11C, 11D Control unit
13 Storage unit
105 Sensor
109 Display apparatus
110 First representative value acquisition unit
111 Second representative value acquisition unit
115C, 115D Availability determination unit
116 Stamping time monitoring unit
131 Temporary storage unit
132 Log storage unit
200 Sensor terminal
201 Sensor
300, 300B, 300C, 300D Relay terminal
301 Data reception unit
304, 304B, 304C, 304D Control unit
305, 305B, 305C, 305D First representative value acquisition unit
306 Second representative value acquisition unit
309 Availability determination unit
310 Stamping time monitoring unit
400 External terminal
401 Data reception unit
3021 Temporary storage unit
3022 Log storage unit
D1 to D15 Time-series data

1. A biological information analysis system comprising:
a data receiver configured to receive time-series data of biological information from a sensor terminal having a biological sensor configured to acquire biological information;
a storage configured to store the received time-series data;
a first representative value acquisitor configured to acquire a first representative value based on data in a predetermined period among the received time-series data; and
a second representative value acquisitor configured to acquire a second representative value based on a plurality of first representative values consecutive on a time axis, the first representative value being one of the plurality of first representative values,
wherein, in a case where an error occurs in communication between the data receiver and the sensor terminal and a predetermined condition is satisfied, the first representative value acquisitor uses pre-error time-series data transmitted before the error occurs and stored in the storage and post-error time-series data transmitted from the sensor terminal after the error occurs, to acquire the first representative value.

2. The biological information analysis system according to claim 1,
wherein the storage further includes a temporary storage configured to temporarily store the pre-error time-series data, and
when the error occurs in the communication between the data receiver and the sensor terminal, the first representative value acquisitor uses the pre-error time-series data stored in the temporary storage and the post-error time-series data transmitted from the sensor terminal after the error occurs, to acquire the first representative value.

3. The biological information analysis system according to claim 1, further comprising:
an availability determinator configured to determine whether the pre-error time-series data is used for acquisition of the first representative value,
wherein the first representative value acquisitor acquires the first representative value by using the pre-error time-series data when the availability determinator determines that the pre-error time-series data is used for acquisition of the first representative value.

4. The biological information analysis system according to claim 3, further comprising:
a log storage configured to record log information including execution content and an execution time of a procedure in the data receiver in reception of the time-series data from the sensor terminal,
wherein the availability determinator determines whether the pre-error time-series data is used for acquisition of the first representative value, based on the log information stored in the log storage.

5. The biological information analysis system according to claim 3, further comprising:
a stamping time monitor configured to monitor a time when the time-series data is acquired by the sensor terminal or a time when the time-series data arrives at the data receiver,
wherein the availability determinator determines whether the pre-error time-series data is used for acquisition of the first representative value, based on a monitoring result of the time in the stamping time monitor.

6. The biological information analysis system according to claim 1, wherein the biological information includes at least one of an electrocardiographic waveform, heart rate, a pulse wave, blood pressure, bioimpedance, myoelectric potential, respiration, moving speed, acceleration, angular velocity, atmospheric pressure, light, temperature, or humidity.

7. A non-transitory computer readable medium having a program stored therein for causing a computer to function as the biological information analysis system according to claim 1.

8. A biological information analysis method comprising:
receiving time-series data of biological information from a sensor terminal having a biological sensor configured to acquire biological information;
storing the received time-series data;
acquiring a first representative value based on data in a predetermined period among the received time-series data; and
acquiring a second representative value based on a plurality of first representative values consecutive on a time axis, the first representative value being one of the plurality of first representative values,
wherein, in the acquiring of the first representative value, in a case where a predetermined condition is satisfied when an error occurs in communication between the data receiver and the sensor terminal, the first representative value is acquired using pre-error time-series data transmitted before the error occurs and stored in the storage and post-error time-series data transmitted from the sensor terminal after the error occurs.
